(19) United States
(12) Patent Application Publication
(10) Pub. No.: US 2023/0340031 A1
(43) Pub. Date: Oct. 26, 2023

(54) SELF-ASSEMBLING PROTEIN NANOPARTICLES WITH BUILT-IN SIX-HELIX BUNDLE PROTEINS

(71) Applicant: ALPHA-O PEPTIDES AG, Riehen (CH)

(72) Inventors: Caroline KULANGARA, Basel (CH); Sara Maria PAULILLO, Basel (CH); Matteo PIAZZA, Chester (GB); Senthil Kumar RAMAN, Basel (CH); Peter BURKHARD, Schopfheim (DE)

(21) Appl. No.: 18/189,601

(22) Filed: Mar. 24, 2023

Related U.S. Application Data
(63) Continuation of application No. 16/495,590, filed on Sep. 19, 2019, now abandoned.

(30) Foreign Application Priority Data
Mar. 23, 2017 (EP) 17162540.3

(51) Int. Cl. C07K 14/005; A61K 39/21; A61K 39/145

(52) U.S. Cl. CPC C07K 14/005; A61K 39/145; A61K 39/21; A61K 2039/55555

(57) ABSTRACT

The present invention relates to self-assembling protein nanoparticles with built-in six-helix bundle proteins. Proteins or peptides comprising a loop region are stabilized by attaching them to six-helix bundle (SHB) proteins and integrating them into self-assembling protein nanoparticles (SAPNs).

### FIELD OF THE INVENTION

The present invention relates to self-assembling protein nanoparticles with built-in six-helix bundle proteins. Proteins or peptides comprising a loop region are stabilized by attaching them to six-helix bundle (SHB) proteins and integrating them into self-assembling protein nanoparticles (SAPNs).

### BACKGROUND OF THE INVENTION

The surface proteins of enveloped viruses are critically important in the early state of virus infection. For example, in immunodeficiency viruses (HIV in humans, SlV in simians) they mediate direct fusion of the viral envelope with the cellular membrane after docking of the virus to the cell surface. Similar structural changes occur in the influenza virus hemagglutinin (HA) protein and it has been postulated that large-scale structural rearrangements of HA in influenza or glycoprotein 160 (gp160) in HIV are the reason for the transition of the metastable native (pre-fusogenic) state to a stable fusion-active (fusogenic) state for many of the enveloped virus proteins. The extracellular domains of these proteins exhibit domain organizations with several features that are characteristic and which likely determine their function during activation of retroviral membrane fusion. These proteins usually consist of an N-terminal stretch, followed by two heptad repeats, separated by disulfide containing loop structures. These loops structures may be very large and contain a fully folded domain such as the head domain of HA. Close to the N-terminal end a hydrophobic stretch is located (fusion peptide), which is thought to be inserted into the cellular membrane at an early stage in the fusion process. These proteins contain two regions with a seven amino acid hydrophobic repeat (heptad-repeat) the key signature of coiled coil structures.

In the case of HIV during the early stages of the membrane fusion process, the trimeric envelope glycoprotein contains gp41 (as part of gp160) in its pre-fusogenic conformation. Following binding to the receptor CD4 and followed by the binding to the co-receptor CXCR5/CCR4, a transient species of gp41, the so-called pre-hairpin intermediate, is formed exposing the fusion-peptide region and at the same time the N-terminal coiled-coil trimer is formed. The fusion-active hairpin structure is then formed by the association of the C-terminal heptad-repeat region with the trimeric N-terminal coiled coil and leads to apposition of viral and cellular membranes (Pancera, M., et al., Nature 2014, 514(7523): 455-461).

It is known that conformation-specific display of B-cell epitopes is crucial for the induction of protective immune responses. Such an immune response is characterized by the production of conformation-specific antibodies that readily recognize the antigen of interest with high specificity.

Proper conformation of the B-cell epitope is dependent on proper folding or refolding of the protein. Various methods have been used to display surface glycoproteins in their native conformation. Mostly, the attempt is to stabilize the glycoprotein trimer by attaching a trimeric protein domain such as a coiled coil or the foldon domain of fibritin (Guthe, S., et al. J Mol Biol 2004, 337(4): 905-915) to the molecule of interest. This has been shown for the HA molecule of influenza in which proper folding and hence conformation-specific display of the HA stem domain was accomplished by attachment of HA to the foldon domain (Lu, Y., et al. Proc Natl Acad Sci USA 2014, 111(1): 125-130.)

Using the intrinsic trimeric symmetry of ferritin nanoparticles, Kanekiyo et al. have demonstrated that HA is properly folded when engineered onto this nanoparticulate system (Kanekiyo, M., et al. Nature 2013, 499(7456): 102-106.) In an elaborate experimental approach, the SHB of HIV has been used to design HA-intermediates to figure out the best stem design of HA. In this approach the architecture of the HA-intermediates can be described as B1 - L1 - SHB1 - L2 - SHB2 - L3 - B2, i.e. the B-cell epitope does not form a loop structure, but rather the SHB is built-in into the B cell epitope, which thus is split into two separate fragments B1 and B2. Also, the SHB is not part of the final stem design of the HA immunogen used for vaccination (Yassine, H. M., et al. Nat Med 2015, 21(9): 1065-1070).

Further, stabilization of the RSV F protein by an SHB has been demonstrated (WO 2014/079842 A1). In this approach the two helices of the SHB are on separate polypeptide chains.

Proper refolding of viral trimeric glycoproteins can usually only be accomplished in a eukaryotic protein expression system. Loop-formation during refolding is critical for correct conformation of the metastable glycoproteins of enveloped viruses, which has been demonstrated for HA (Daniels, R., et al. Mol Cell 2003, 11(1): 79-90). Loop-formation is naturally achieved on the ER membrane during eukaryotic protein expression, where HA is held in a loop conformation during protein synthesis and protein folding (Daniels, R., et al. Mol Cell 2003, 11(1): 79-90).

It has now surprisingly been found that - if the oligomeric protein such as e.g. a trimeric protein forms a loop structure, i.e. the N-terminus and the C-terminus of the protein are in close proximity - then instead of using a simple oligomeric domain, an SHB can be used to improve the stabilization of the loop-forming protein. Thus, instead of using a simple trimeric coiled-coil domain or the foldon domain of fibritin only on one terminus, the loop-forming protein can be stabilized by attaching both of its ends (i.e. the N-terminus and the C-terminus) to the ends of the two helices of an SHB. As an example, influenza HA can be attached with its N- and C-terminus to the SHB of the HIV gp41, thus locking it in its metastable pre-fusion conformation. Such an SHB with a built-in trimeric B-cell epitope can then be engineered into the architecture of SAPNs, thus generating a novel type of SAPN backbone.

This novel type of nanoparticle backbone is ideally suited as a scaffold to present proteins that are folded in a loop structure (i.e. the N- and the C-terminus of the protein are in close proximity to each other) on the surface of the nanoparticle. Such a nanoparticle scaffold allows to stabilize the loop-structured protein in its native conformation. Of particular interest are loop-structured proteins that form trimers. It is of high interest that many of the surface proteins of enveloped viruses have exactly such a trimeric loop structure. Examples are the influenza HA, the gB protein of CMV, the F protein of RSV, the gp160 of HIV and many more. These trimeric surface proteins of enveloped viruses are in a metastable pre-fusogenic state that can be stabilized by engineering it on the helix-loop-helix motif of the SHB within the nanoparticles of the present invention. Alternatively, substructures of trimeric proteins can be held together in trimeric conformation using the SHB-SAPN as a scaffold. Also simple loop structures can be displayed as loops on the SHB-SAPN without the need and emphasis to form a particular trimeric conformation but simply to be restrained into a loop structure.

The SHB-SAPNs of this invention offer a very elegant way to display loop-forming peptides and proteins in their native conformation. The B-cell epitopes as loop-forming peptides and proteins can be very simple such as β-turn peptides but they can also be very complex structures like the trimeric surface glycoproteins of enveloped viruses.

### SUMMARY OF THE INVENTION

The invention relates to a self-assembling protein nanoparticle (SAPN) consisting of a multitude of building blocks of formula (la) or (lb)

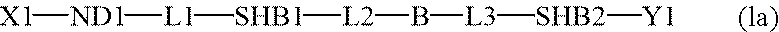

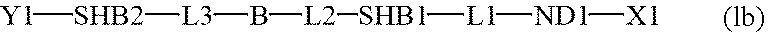

consisting of a continuous chain comprising an oligomerization domain ND1, a linker L1, a domain SHB1, a linker L2, a domain B comprising a loop region, a linker L3, a domain SHB2, and further substituents X1 and Y1, wherein
ND1 is a peptide or protein that comprises oligomers (ND1)ₘ of m subunits ND1,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X1 is absent or a peptide or protein sequence comprising 1 to 1000 amino acids that may be further substituted,
Y1 is absent or a peptide or protein sequence comprising 1 to 1000 amino acids that may be further substituted,
wherein the multitude of building blocks of formula (la) or formula (lb) is optionally co-assembled with a multitude of building blocks of formula (lla) or formula (llb)

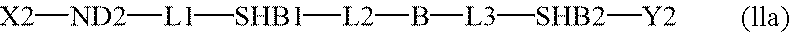

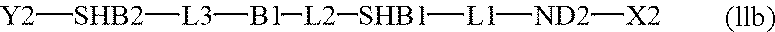

consisting of a continuous chain comprising an oligomerization domain ND2, a linker L1, a domain SHB1, a linker L2, a domain B comprising a loop region, a linker L3, a domain SHB2, and further substituents X2 and Y2, wherein
ND2 is a peptide or protein that comprises oligomers (ND2)ₘ of m subunits ND2,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X2 is absent or a peptide or protein sequence comprising 1 to 1000 amino acids that may be further substituted,
Y2 is absent or a peptide or protein sequence comprising 1 to 1000 amino acids that may be further substituted,
and wherein at least one of X2 and Y2 of formula (lla) and/or formula (llb)is different from X1 and Y1 of formula (la) and/or formula (lb).

### BRIEF DESCRIPTION OF THE FIGURES

FIG. 1: Schematic diagram of the monomer forming an SHB nanoparticle.

The following are the building blocks of the monomer:
SHB1 is one of the two peptides or proteins forming an SHB
B is a protein comprising a loop region, preferentially a monomer of a trimer
SHB2 is the other of the two peptides or proteins forming an SHB protein
ND1 is a protein that forms oligomers (ND1)ₘ of m subunits ND1
L1, L2 and L3 are linkers connecting ND1, SHB1, B and SHB2
X1 and Y1 are peptide or protein sequences at either end of the monomer

FIG. 2: Molecular model of HC_AD1g.

Molecular model of the monomer (A), trimer (B) and icosahedral particle (C) formed by a protein string with the architecture X1 - ND1 - L1 - SHB1 - L2 - B - L3 - SHB2 in which Y1 is absent. SHB1 and SHB2 forming the six-helix bundle are indicated by the text. The loop-forming protein is a portion of the gB protein of CMV that forms the trimeric surface-exposed tip of gB, while the SHB is part of the gp41 protein from HIV.

FIG. 3: Transmission electron micrograph of HC_AD1g.

After refolding and co-assembly of recombinantly expressed protein, the sample was adsorbed on carbon-coated grids and negatively stained with 2% uranyl acetate. The nanoparticles have the sequence SEQ ID NO:1 described in Example 1. The bar represents 200 nm.

FIG. 4: Vector map of pPEP-T.

“prom”: promoter; “term”: terminator; “ori”: origin; “bp”: base pairs; “amp”: ampicillin resistance gene.

FIG. 5: SDS-PAGE of the construct HC_AD1g.

This construct has a theoretical molecular weight of 36.0 kDa

A) Expression levels in different cell lines
UI - Uninduced
I - Induced

B) Purity after Ni-affinity purification.

FIG. 6: Computer model of F34-HAPR-HIVlong.

Molecular model of the monomer (A), trimer (B) and icosahedral particle (C) formed by a protein string with the architecture Y1 - SHB2 - L3 - B - L2 - SHB1 - L1 - ND1 - X1. SHB1 and SHB2 forming the six-helix bundle are indicated by the text. The loop-forming protein is HA from influenza that forms the trimeric surface-exposed glycoprotein while the SHB is part of the gp41 protein from HIV. The view in C is down the five-fold symmetry axis of the icosahedron.

FIG. 7: SDS-PAGE of the construct F34-HAPR-HIVlong.

This construct has a theoretical molecular weight of 77.9 kDa

A) Expression levels before and after induction
ui - uninduced
i - induced

B) Purity after Ni-affinity purification.

FIG. 8: Transmission electron micrograph of F34-HAPR-HIVlong.

After refolding and co-assembly of recombinantly expressed protein, the sample was adsorbed on carbon-coated grids and negatively stained with 2% uranyl acetate. The nanoparticles have the sequence SEQ ID NO:15 described in Example 5. The bar represents 100 nm.

FIG. 9: ELISA-analysis of the conformation of the HA molecules on the F34-HAPR-HIVlong particles.

A) Recognition of F34-HAPR-HIVlong and inactivated PR8/34 virus by the mAb IC5-4F8

B) Recognition of F34-HAPR-HIVlong and inactivated PR8/34 virus by the polyclonal hyperimmune serum

C) Loss of PR8/34 recognition by pre-incubation of mAb IC5-4F8 with 80 ng F34-HAPR-HIVlong

D) Loss of PR8/34 recognition by pre-incubation of the polyclonal hyperimmune serum with 80 ng F34-HAPR-HIVlong

Y-axes: relative OD-values from the different ELISA measurements.

FIG. 10: Analysis of the conformation of the HA molecules on the F3-HAPR trimers by ELISA.

Recognition of HA by the polyclonal hyperimmune serum on F3-HAPR and inactivated PR8/34 virus at different protein concentrations of 5 µg/ml (black), 1.7 µg/ml (dotted), 0.56 µg/ml (dashed) and 0.19 µg/ml (white), respectively. The F3-HAPR was stored at different temperature conditions. RT: room temperature.

FIG. 11: Survival rate of immunized mice after challenge with a lethal dose of 100 PFU (10 LD90) of A/PR/8/34 (H1N1).
Δ F34-HAPR-HIVlong
X Inactivated virus PR8/34
□ PBS buffer

FIG. 12: Analysis of the immune response after challenge with PR8/34.

A) Body weight after immunization with F34-HAPR-HIVlong.
Δ Mouse 1
■ Mouse 2
● Mouse 3
X Mouse 4
◇ Mouse 5

B) Antibody titer against the inactivated virus PR8/34 after immunization with F34-HAPR-HIVIong.
Δ Mouse 1
■ Mouse 2
● Mouse 3
X Mouse 4
◇ Mouse 5

FIG. 13: Analysis of the immune response after challenge with PR8/34.

A) Body weight after immunization with inactivated virus PR8/34.
Δ Mouse 6
■ Mouse 7
● Mouse 8
X Mouse 9
◇ Mouse 10

B) Antibody titer against the inactivated virus PR8/34 after immunization with inactivated virus PR8/34.
Δ Mouse 6
■ Mouse 7
● Mouse 8
X Mouse 9
◇ Mouse 10

FIG. 14: Molecular model of 4TVP-1 ENV.

Molecular model of the monomer (A), trimer (B) and icosahedral particle (C) formed by a protein string with the architecture X1 - ND1 - L1 - SHB1 - L2 - B - L3 - SHB2 in which L2 and L3 are peptide bonds and Y1 is absent. SHB1 and SHB2 forming the six-helix bundle are indicated by the text. The loop-forming protein is the V1/V2-loop of the gp120 protein of HIV that forms the trimeric surface-exposed tip of gp120, while the SHB is part of the gp41 protein from HIV.

### DETAILED DESCRIPTION OF THE INVENTION

In the present invention SHBs are described that are built-in, i.e. incorporated into the architecture of known SAPNs such as SAPNs described e.g. by Raman S.K. et al. Nanomed 2006, 2(2): 95-102; Pimentel T. A., et al. Chem Biol Drug Des. 2009. 73(1): 53-61; Indelicato, G., et al. Biophys J. 2016, 110(3): 646-660; Karch, C. P., et al. Nanomedicine 2016, 13(1): 241-251. In order to stabilize loop forming peptides or proteins, preferably proteins with an oligomerization state of three are used herein. SAPNs which can be used as basis to construct the SAPNs of the present invention are also described in WO2004071493, WO2009109428 and WO2015104352.

The invention relates to a self-assembling protein nanoparticle (SAPN) consisting of a multitude of building blocks of formula (la) or (lb)

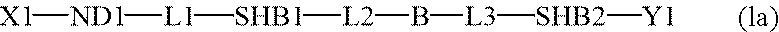

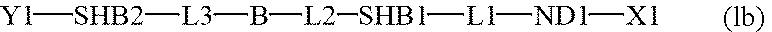

consisting of a continuous chain comprising an oligomerization domain ND1, a linker L1, a domain SHB1, a linker L2, a domain B comprising a loop region, a linker L3, a domain SHB2, and further substituents X1 and Y1, wherein
ND1 is a peptide or protein that comprises oligomers (ND1)ₘ of m subunits ND1,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X1 is absent or a peptide or protein sequence comprising 1 to 1000 amino acids that may be further substituted,
Y1 is absent or a peptide or protein sequence comprising 1 to 1000 amino acids that may be further substituted,
wherein the multitude of building blocks of formula (la) or formula (lb) is optionally co-assembled with a multitude of building blocks of formula (lla) or formula (llb)

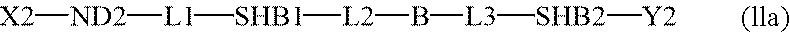

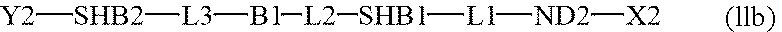

consisting of a continuous chain comprising an oligomerization domain ND2, a linker L1, a domain SHB1, a linker L2, a domain B comprising a loop region, a linker L3, a domain SHB2, and further substituents X2 and Y2, wherein
ND2 is a peptide or protein that comprises oligomers (ND2)ₘ of m subunits ND2,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X2 is absent or a peptide or protein sequence comprising 1 to 1000 amino acids that may be further substituted,
Y2 is absent or a peptide or protein sequence comprising 1 to 1000 amino acids that may be further substituted,
and wherein at least one of X2 and Y2 of formula (lla) and/or formula (llb)is different from X1 and Y1 of formula (la) and/or formula (Ib).

In a preferred embodiment the invention relates to a self-assembling protein nanoparticle (SAPN) consisting of a multitude of building blocks of formula (la) or (lb)

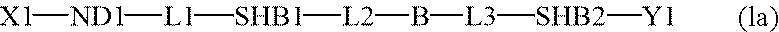

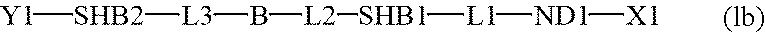

consisting of a continuous chain comprising an oligomerization domain ND1, a linker L1, a domain SHB1, a linker L2, a domain B comprising a loop region, a linker L3, a domain SHB2, and further substituents X and Y, wherein
ND1 is a peptide or protein that comprises oligomers (ND1)m of m subunits ND1,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X1 is absent or a peptide or protein sequence comprising 1 to 1000 amino acids that may be further substituted,
Y1 is absent or a peptide or protein sequence comprising 1 to 1000 amino acids that may be further substituted.

In a further preferred embodiment the invention relates to a self-assembling protein nanoparticle (SAPN) consisting of a multitude of building blocks of formula (la) or (lb)

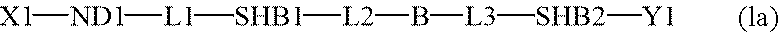

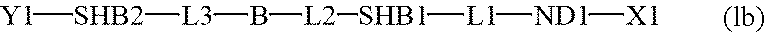

consisting of a continuous chain comprising an oligomerization domain ND1, a linker L1, a domain SHB1, a linker L2, a domain B comprising a loop region, a linker L3, a domain SHB2, and further substituents X1 and Y1, wherein
ND1 is a peptide or protein that comprises oligomers (ND1)ₘ of m subunits ND1,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X1 is absent or a peptide or protein sequence comprising 1 to 1000 amino acids that may be further substituted,
Y1 is absent or a peptide or protein sequence comprising 1 to 1000 amino acids that may be further substituted,

wherein the multitude of building blocks of formula (la) or formula (lb) is co-assembled with a multitude of building blocks of formula (lla) or formula (llb)

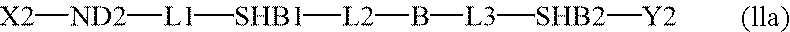

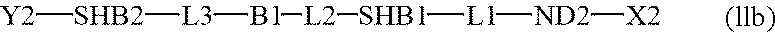

consisting of a continuous chain comprising an oligomerization domain ND2, a linker L1, a domain SHB1, a linker L2, a domain B comprising a loop region, a linker L3, a domain SHB2, and further substituents X2 and Y2, wherein
ND2 is a peptide or protein that comprises oligomers (ND2)ₘ of m subunits ND2,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X2 is absent or a peptide or protein sequence comprising 1 to 1000 amino acids that may be further substituted,
Y2 is absent or a peptide or protein sequence comprising 1 to 1000 amino acids that may be further substituted,
and wherein at least one of X2 and Y2 of formula (lla) and/or formula (llb)is different from X1 and Y1 of formula (la) and/or formula (lb).

In case a multitude of building blocks of formula (la) or formula (lb) co-assembles with a multitude of building blocks of formula (lla) or formula (llb),normally a building block of formula (la) co-assembles with a building block of formula (lla) and a building block of formula (lb) co-assembles with a building block of formula (llb). In a preferred embodiment the oligomerization domain ND1, the linker L1, the domain SHB1, the linker L2, the domain B comprising a loop region, the linker L3, and the domain SHB2 of formula (la) or formula (lb) are identical to the oligomerization domain ND2, the linker L1, the domain SHB1, the linker L2, the domain B comprising a loop region, the linker L3, and the domain SHB2 of formula (lla) or formula (llb).

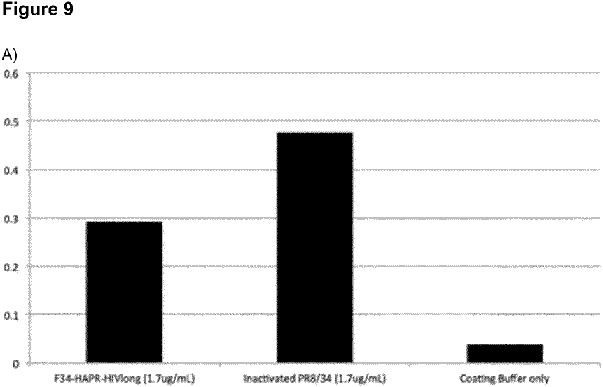

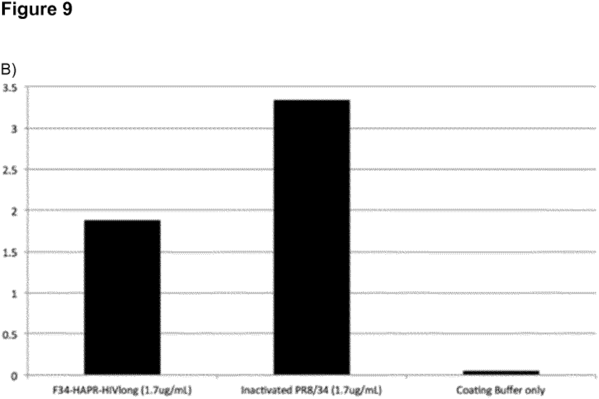

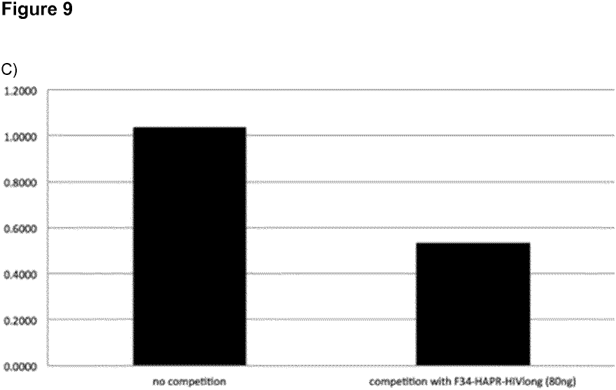

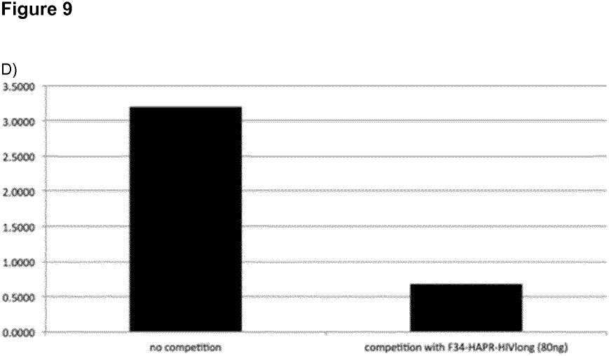

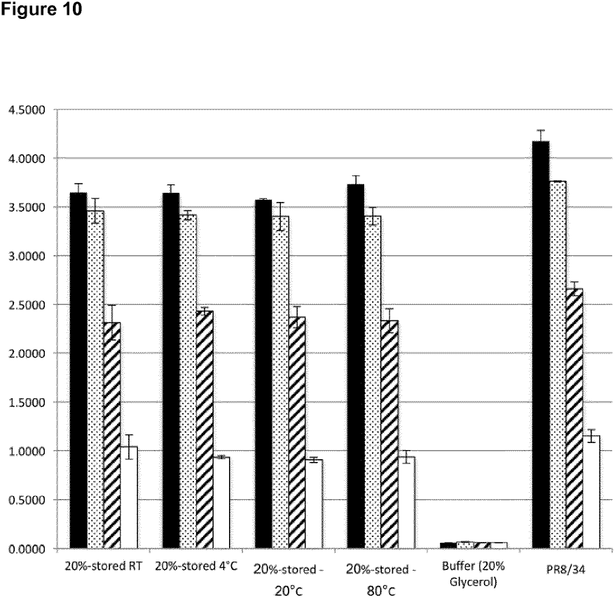

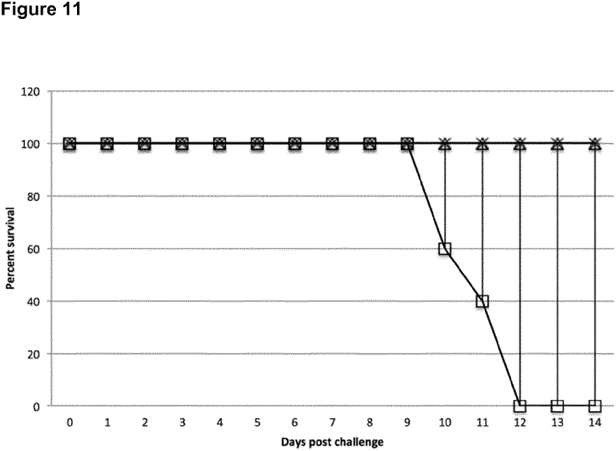

In the present invention engineering the N- and C-termini of proteins such as glycoproteins on the two helices of an SHB that is part of the SAPN architecture restrains the B-cell epitope into a loop conformation during refolding. This is critical and allows the protein to be correctly refolded from denaturing conditions surprisingly even after production in a prokaryotic expression system. Hence, eukaryotic expression is not necessarily needed for proper refolding of the protein. For refolding it is important that a loop is formed which holds the N-terminus and the C-terminus of the protein in close proximity as provided by the SHB-SAPNs of the present invention. Proper refolding of bacterially expressed HA from denaturing conditions using the present invention is demonstrated by recognition and binding of conformation-specific by mAbs and hyperimmune serum to the SHB-SAPN-based HA immunogen (FIGS. 9 and 10).

### Monomeric Building Blocks

A peptide (or polypeptide or protein) is a chain or sequence of amino acids covalently linked by amide bonds. The peptide may be natural, modified natural, partially synthetic or fully synthetic. Modified natural, partially synthetic or fully synthetic is understood as meaning not occurring in nature. The term amino acid embraces both naturally occurring amino acids selected from the 20 essential natural α-L-amino acids, synthetic amino acids, such as α-D-amino acids, 6-aminohexanoic acid, norleucine, homocysteine, or the like, as well as naturally occurring amino acids which have been modified in some way to alter certain properties such as charge, such as phoshoserine or phosphotyrosine, or other modifications such as n-octanoyl-serine, or the like. Derivatives of amino acids are amino acids in which for example the amino group forming the amide bond is alkylated, or a side chain amino-, hydroxyl- or thio-group is alkylated or acylated, or a side chain carboxy-group is amidated or esterified. Preferably a peptide or protein of the invention comprises amino acids selected from the 20 essential natural α-L-amino acids.

In a rough approximation, peptides can be distinguished from proteins on the basis of their size, i.e. approximately a chain of 50 amino acids or less can be considered to be a peptide, while longer chains can be considered to be proteins. Thus, the term “peptide” as used herein refers to an amino acid chain of 50 amino acids or less, preferably to an amino acid chain of 2 to 50 amino acids, the term “protein” as used herein refers to an amino acid chain of more than 50 amino acids, preferably to an amino acid chain of 51 to 10000 amino acids. Dipeptides are the shortest peptides and consist of 2 amino acids joined by a single peptide bond. Likewise, tripeptides consist of three amino acids, tetrapeptides consist of four amino acids, etc. A polypeptide is a long, continuous, and unbranched peptide chain. In the literature boundaries of the size that distinguish peptides from proteins are somewhat weak. Sometimes long “peptides” such as amyloid beta have been considered proteins, and vice versa smaller proteins such as insulin have been referred to as peptides.

Oligomerization domains according to the invention are preferably coiled coils. A coiled coil is a protein sequence with a contiguous pattern of mainly hydrophobic residues spaced 3 and 4 residues apart, which assembles to form a multimeric bundle of helices, as will be explained in more detail herein below.

All components (X1, X2, ND1, ND2, L1, SHB1, L2, B, L3, SHB2, Y1 and Y2) of the monomeric building block(s) may optionally be further substituted by targeting entities, or substituents reinforcing the adjuvant properties of the nanoparticle. Substituted means a replacement of one chemical group on the monomeric building block by another chemical group yielding a substituent that is covalently linked to the monomeric building block. Such substituents may be an immunostimulatory nucleic acid, preferably an oligodeoxynucleotide containing deoxyinosine, an oligodeoxynucleotide containing deoxyuridine, an oligodeoxynucleotide containing a CG motif, CpGs, imiquimod, resiquimod, gardiquimod, an inosine and cytidine containing nucleic acid molecule, or the like. A particular targeting entity considered as substituent is an ER-targeting signal, i.e. a signal peptide that induces the transport of a protein or peptide to the endoplasmic reticulum (ER).

In a preferred embodiment, the building blocks of formula (la) or (lb) comprises either substituent X1 or substituent Y1 and/or the building blocks of formula (lla) or (llb)comprises either substituent X2 or substituent Y2.

In another preferred embodiment, the building blocks of formula (la) or (lb) comprises substituents X1 and Y1 and/or the building blocks of formula (lla) or (llb) comprises substituent X2 and Y2. Thus in a most preferred embodiment the substituent is a peptide or protein substituent and is termed X1, X2, Y1 or Y2 representing an extension of the protein chain, e.g. as X1 - ND1 - L1 - SHB1 - L2 - B - L3 - SHB2 - Y1 or X2 - ND2 - L1 - SHB1 - L2 - B - L3 - SHB2 - Y2 usually at one end, preferably at both ends to generate a combined single continuous protein sequence. Conveniently, such a single continuous protein chain may be expressed in a recombinant protein expression system as one single molecule. Substituents X1, Y1, X2 and Y2 independently from each other are a peptide or a protein sequence comprising 1 to 1000 amino acids preferably sequences corresponding to fully folded proteins or protein domains to be used either as B-cell epitopes, or flagellin or a subset of its four domains as described in WO2015104352 to enhance the immune response.

Flagellin has a molecular architecture that is composed of four domains D0, D1, D2 and D3. The protein chain starts with the N-terminus in the D0 domain and runs in a big loop through the other domains D1, D2 and D3 to the tip of the molecule where it turns and runs back through D3, D2 and D1 to bring its C-terminal end in the D0 domain very close to the N-terminal end. Flagellin has two modes of activation of the innate immune system. The first mode is by binding to the TLR5 receptor mainly through a highly conserved portion of its D1 domain (Yoon S.I. et al., Science 2012, 335:859-64). The other mode of activation is by interaction with the inflammasome mainly through a highly conserved C-terminal portion of its D0 domain (Lightfield K.L. et al., Nat Immunol. 2008, 9:1171-8).

Thus in a preferred embodiment at least one of the substituents X1, Y1, X2 and Y2 is a full length flagellin e.g. a full length Salmonella typhimurium flagellin or a flagellin comprising only two or three domains, preferably a flagellin comprising at least the TLR5 binding domain D1 more preferably a flagellin comprising the D0 and D1 domains, in particular the flagellin comprising the sequence MAQVINTNSLSLLTQNNLNKSQSALGTAIERLSSGLRINSAKDDA AGQAIANRFTANIKGLTQASRNANDGISIAQTTEGALNEINNNLQRVRELAVQSANSTNSQS DLDSIQAEITQRLNEIDRVSGQTQFNGVKVLAQDNTLTIQVGANDGETIDIDLKQINSQTLGLD SLNVHGAPVDPASPWTENPLQKlDAALAQVDALRSDLGAVQNRFNSAITNLGNTVNNLSEA RSRIEDSDYATEVSNMSRAQILQQAGTSVLAQANQVPQNVLSLLR (SEQ ID NO:37) or the sequence MAQVINTNSLSLLTQNNLNRSQSALGTAIERLSSGLRINSARDDAAGQAIANRFT ANIRGLTQASRNANDGISIAQTTEGALNEINNNLQRVRELAVQSANSTNSQSDLDSIQAEITQ RLNEIDRVSGQTQFNGVRVLAQDNTLTIQVGANDGETIDIDLRQINSQTLGLDQLNVQQKYK DGDKGDDKTENPLQRIDAALAQVDALRSDLGAVQNRFNSAITNLGNTVNNLSEARSRIEDSD YATEVSNMSRAQILQQAGTSVLAQANQVPQNVLSLLR (SEQ ID NO:38). The missing domain(s) may be substituted by a flexible linker segment of 1 to 20 amino acids joining the two ends of the remaining flagellin sequence, or they may be replaced by a fully folded protein antigen. In a preferred embodiment the missing domain(s) are substituted by the flexible linker comprising the amino acid sequence QLNVQQKYKDGDKGDDKTENPLQ (SEQ ID NO:39). The flexible linker region may contain suitable attachment sites for the covalent coupling of antigens. Thus, a flagellin derivative construct lacking the D2 and D3 domains of flagellin can easily be engineered, simply by connecting the protein chain at the interface of the D1 and D2 domains. Similar, the tip domains (either D3, or D2 and D3 together) can be replaced by a protein antigen, provided this protein antigen with its N- and C-termini can be connected to the N- and C-termini at the interface between D1 and D2. The tip domains D2 and D3 can also be replaced by a peptide sequence with suitable residues for the covalent coupling of antigen molecules.

In another preferred embodiment X1, Y1, X2 and Y2 independently from each other may also comprise a string of one or more CD4 and/or CD8 epitopes. In another preferred embodiment X1, Y1, X2 and Y2 independently from each other may comprise a combination of one or more of these types of immunological relevant CD4/CD8 peptide and protein sequences.

In another preferred embodiment the multitude of building blocks of formula (la) or formula (lb) is co-assembled with a multitude of building blocks of formula (lla) or formula (llb), wherein at least one of X2 and Y2 of formula (lla) and/or formula (llb),preferably one of X2 and Y2 of formula (lla) and/or formula (llb),is a full length flagellin or a flagellin comprising only two or three domains, preferably a flagellin comprising the D0 and D1 domains, in particular the flaggellin as shown in SEQ ID NO:37 and/or SEQ ID NO:38.

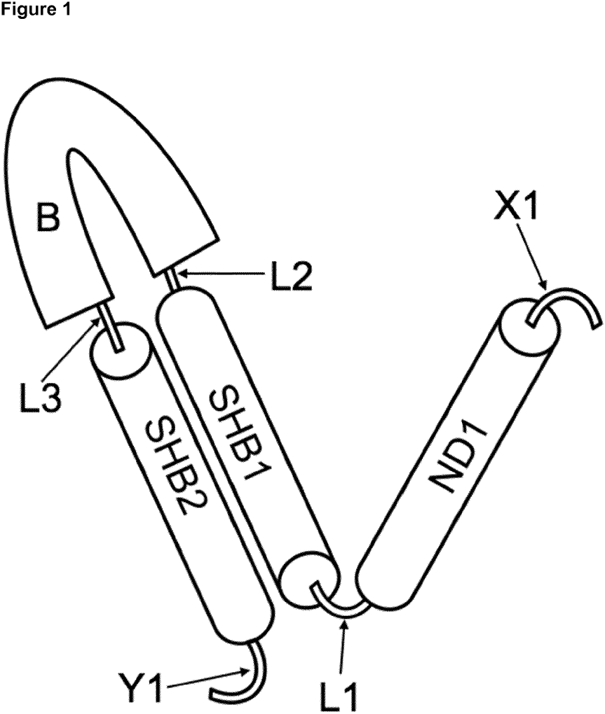

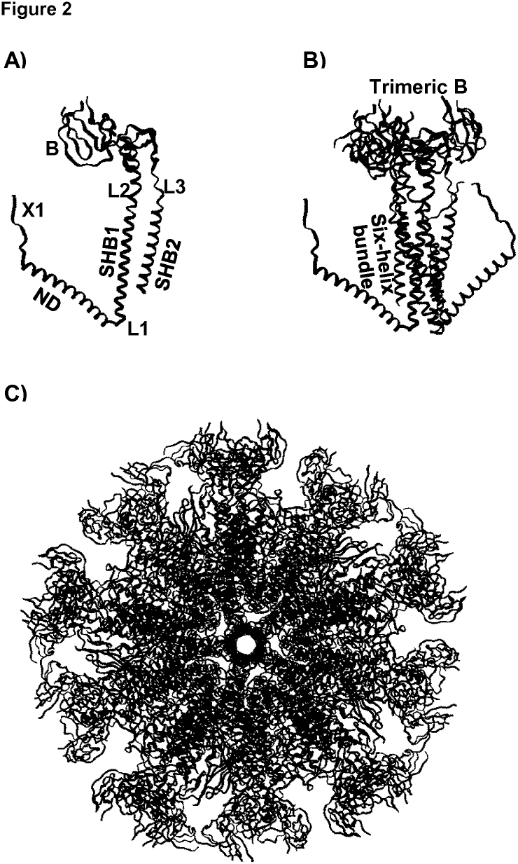

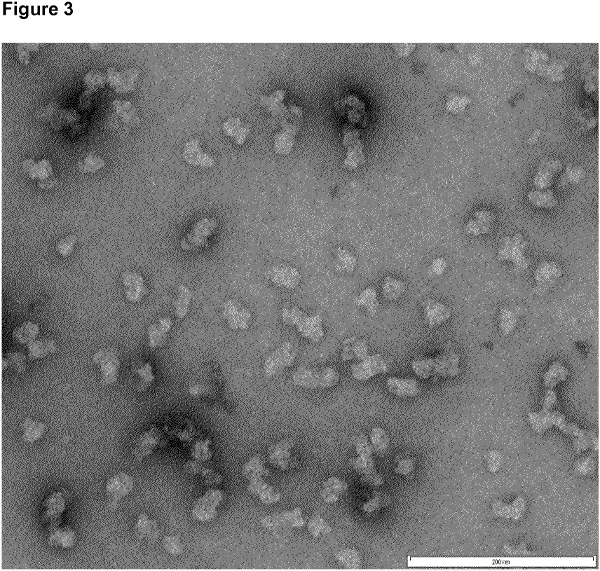

If Y1 and Y2 are attached to the SHB-domain, this attachment site of the SHB is pointing towards to core of the SAPN (see FIGS. 1 and 2), flagellin is preferably attached to the ND1 and/or ND2 domain. Thus in a preferred embodiment X1 and/or X2 is a full length flagellin e.g. a full length Salmonella typhimurium flagellin or a flagellin comprising only two or three domains, preferably a flagellin comprising at least the TLR5 binding domain D1 more preferably a flagellin comprising the D0 and D1 domains, in particular the flagellin with comprising the sequence MAQVINTNSLSLLTQNNLNKSQSALGTAIERLSSGLRINSAKDD AAGQAIANRFTANIKGLTQASRNANDGISIAQTTEGALNEINNNLQRVRELAVQSANSTNSQS DLDSIQAEITQRLNEIDRVSGQTQFNGVKVLAQDNTLTIQVGANDGETIDIDLKQINSQTLGLD SLNVHGAPVDPASPWTENPLQKIDAALAQVDALRSDLGAVQNRFNSAITNLGNTVNNLSEA RSRIEDSDYATEVSNMSRAQILQQAGTSVLAQANQVPQNVLSLLR (SEQ ID NO:37) or the sequence MAQVINTNSLSLLTQNNLNRSQSALGTAIERLSSGLRINSARDDAAGQAIANRFT ANIRGLTQASRNANDGISIAQTTEGALNEINNNLQRVRELAVQSANSTNSQSDLDSIQAEITQ RLNEIDRVSGQTQFNGVRVLAQDNTLTIQVGANDGETIDIDLRQINSQTLGLDQLNVQQKYK DGDKGDDKTENPLQRIDAALAQVDALRSDLGAVQNRFNSAITNLGNTVNNLSEARSRIEDSD YATEVSNMSRAQILQQAGTSVLAQANQVPQNVLSLLR (SEQ ID NO:38).

A tendency to form oligomers means that such proteins can form oligomers depending on the conditions, e.g. under denaturing conditions they are monomers, while under physiological conditions they may form, for example, dimers, trimers, tetramers or pentamers. Under predefined conditions they adopt one single oligomerization state, which is needed for nanoparticle formation. However, their oligomerization state may be changed upon changing conditions, e.g. from trimers to dimers upon decreasing salt concentration (Burkhard P. et al., Protein Science 2000, 9:2294-2301) or from pentamers to monomers upon decreasing pH.

A building block architecture according to formula (la) or (lb) and/or formula (lla) or (llb)is clearly distinct from viral capsid proteins. Viral capsids are composed of either one single protein, which forms oligomers of 60 or a multiple thereof, as e.g. the hepatitis virus B particles (EP 1 262 555, EP 0 201 416), or of more than one protein, which co-assemble to form the viral capsid structure, which can adopt also other geometries apart from icosahedra, depending on the type of virus (Fender P. et al., Nature Biotechnology 1997, 15:52-56). SAPNs of the present invention are also clearly distinct from virus-like particles, as they (a) are constructed from other than viral capsid proteins and (b) that the cavity in the middle of the nanoparticle is too small to accommodate the DNA/RNA of a whole viral genome.

Protein oligomerization domains are well-known (Burkhard P. et al., Trends Cell Biol 2001, 11:82-88). In the present invention the oligomerization domain ND1 or ND2 is preferably a coiled-coil domain. A coiled coil is a protein sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled coils with sequences including some irregular distribution of the 3 and 4 residues spacing are also contemplated. Hydrophobic residues are in particular the hydrophobic amino acids Val, lle, Leu, Met, Tyr, Phe and Trp. Mainly hydrophobic means that at least 50% of the residues must be selected from the mentioned hydrophobic amino acids.

### HEPTAD REPEATS AND COILED COILS

For example, in a preferred monomeric building block of formula (la) or (lb) and/or formula (lla) or (llb),ND1 and/or ND2, preferably ND1 and ND2, comprises a heptad repeat or an undecad repeat, more preferably a heptad repeat, in particular a protein of any of the formulae

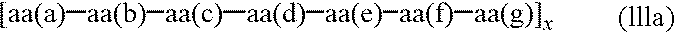

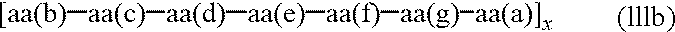

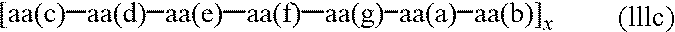

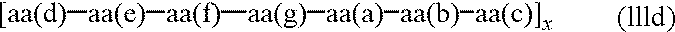

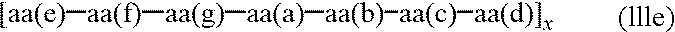

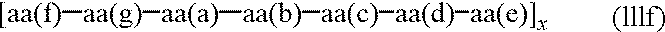

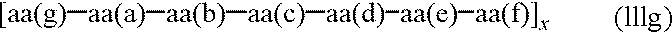

wherein aa means an amino acid or a derivative thereof, aa(a), aa(b), aa(c), aa(d), aa(e), aa(f), and aa(g) are the same or different amino acids or derivatives thereof, preferably aa(a) and aa(d) are the same or different hydrophobic amino acids or derivatives thereof; and x is a figure between 2 and 20, preferably between 3 and 10.

A heptad is a heptapeptide of the formula aa(a)-aa(b)-aa(c)-aa(d)-aa(e)-aa(f)-aa(g) (llla) or any of its permutations of formulae (lllb) to (lllg).

Preferred are monomeric building blocks of formula (la) or (lb) and/or formula (lla) or (llb) wherein the protein oligomerization domain ND1 and/or ND2, preferably ND1 and ND2, comprises
(1) a protein of any of the formulae (llla) to (lllg) wherein x is 3, and aa(a) and aa(d) are selected from the 20 natural α-L-amino acids such that the sum of scores from Table 1 for these 6 amino acids is at least 14, and such proteins comprising up to 17 further heptads; or
(2) a protein of any of the formulae (llla) to (lllg) wherein x is 3, and aa(a) and aa(d) are selected from the 20 natural α-L-amino acids such that the sum of scores from Table 1 for these 6 amino acids is at least 12, with the proviso that one amino acid aa(a) is a charged amino acid able to form an inter-helical salt bridge to an amino acid aa(d) or aa(g) of a neighboring heptad, or that one amino acid aa(d) is a charged amino acid able to form an inter-helical salt bridge to an amino acid aa(a) or aa(e) of a neighboring heptad, and such proteins comprising up to two further heptads. A charged amino acid able to form an inter-helical salt bridge to an amino acid of a neighboring heptad is, for example, Asp or Glu if the other amino acid is Lys, Arg or His, or vice versa.

TABLE 1

Scores of amino acid for determination of preference (coiled-coil propensity)

| Amino acid | Position aa(a) | Position aa(d) |
|---|---|---|
| L (Leu) | 3.5 | 3.8 |
| M (Met) | 3.4 | 3.2 |
| I (Ile) | 3.9 | 3.0 |
| Y (Tyr) | 2.1 | 1.4 |
| F (Phe) | 3.0 | 1.2 |
| V (Val) | 4.1 | 1.1 |
| Q (Gln) | -0.1 | 0.5 |
| A (Ala) | 0.0 | 0.0 |
| W (Trp) | 0.8 | -0.1 |
| N (Asn) | 0.9 | -0.6 |
| H (His) | -1.2 | -0.8 |
| T (Thr) | 0.2 | -1.2 |
| K (Lys) | -0.4 | -1.8 |
| S (Ser) | -1.3 | -1.8 |
| D (Asp) | -2.5 | -1.8 |
| E (Glu) | -2.0 | -2.7 |
| R (Arg) | -0.8 | -2.9 |
| G (Gly) | -2.5 | -3.6 |
| P (Pro) | -3.0 | -3.0 |
| C (Cys) | 0.2 | -1.2 |

Also preferred are monomeric building blocks of formula (la) or (lb) and/or formula (lla) or (llb) wherein the protein oligomerization domain ND1 and/or ND2, preferably ND1 and ND2, comprises a protein selected from the following preferred proteins:
(11) Protein of any of the formulae (llla) to (lllg) wherein aa(a) is selected from Val, lle, Leu and Met, and a derivative thereof, and aa(d) is selected from Leu, Met, Val and lle, and a derivative thereof.
(12) Protein of any of the formulae (llla) to (lllg) wherein one aa(a) is Asn and the other aa(a) are selected from Asn, lle and Leu, and aa(d) is Leu. Such a protein is usually a dimerization domain.
(13) Protein of any of the formulae (llla) to (lllg) wherein aa(a) and aa(d) are both Trp. Such a protein is usually a pentamerization domain.
(14) Protein of any of the formulae (llla) to (lllg) wherein aa(a) and aa(d) are both Phe. Such a protein is usually a tetramerization domain.
(15) Protein of any of the formulae (llla) to (lllg) wherein aa(a) and aa(d) are both either Trp or Phe. Such a protein is usually a pentamerization domain.
(16) Protein of any of the formulae (llla) to (lllg) wherein aa(a) is either Leu or lle, and one aa(d) is Gln and the other aa(d) are selected from Gln, Leu and Met. Such a protein has the potential to be a pentamerization domain.
Other preferred proteins are proteins (1), (2), (11), (12), (13), (14), (15) and (16) as defined hereinbefore, and wherein further
(17) at least one aa(g) is selected from Asp and Glu and aa(e) in a following heptad is Lys, Arg or His; and/or
(18) at least one aa(g) is selected from Lys, Arg and His, and aa(e) in a following heptad is Asp or Glu, and/or
(19) at least one aa(a to g) is selected from Lys, Arg and His, and an aa(a to g) 3 or 4 amino acids apart in the sequence is Asp or Glu. Such pairs of amino acids aa(a to g) are, for example aa(b) and aa(e) or aa(f).

Coiled-coil prediction programs such as PCOILS (http://toolkit.tuebingen.mpg.de/pcoils; Gruber M. et al., J. Struct. Biol. 2006, 155(2): 140-5) or MULTICOIL (http://groups.csail.mit.edu/cb/multicoil/cgi-bin/multicoil.cgi) can predict coiled-coil forming protein sequences. Therefore, in a monomeric building block of formula (la) or (lb) and/or formula (lla) or (llb) ND1 and/or ND2, preferably ND1 and ND2, comprises a protein that contain at least a sequence two heptad-repeats long that is predicted by the coiled-coil prediction program PCOILS to form a coiled-coil with higher probability than 0.9 for all its amino acids with at least one of the window sizes of 14, 21, or 28.

In a more preferred monomeric building block of formula (la) or (lb) and/or formula (lla) or (llb) ND1 and/or ND2, preferably ND1 and ND2, comprises a protein that contains at least one sequence three heptad-repeats long that is predicted by the coiled-coil prediction program PCOILS to form a coiled-coil with higher probability than 0.9 for all its amino acids with at least one of the window sizes of 14, 21, or 28.

In another more preferred monomeric building block of formula (la) or (lb) and/or formula (lla) or (llb)ND1 and/or ND2, preferably ND1 and ND2, comprises a protein that contains at least two separate sequences two heptad-repeats long that are predicted by the coiled-coil prediction program PCOILS to form a coiled-coil with higher probability than 0.9 for all its amino acids with at least one of the window sizes of 14, 21, or 28.

### The RCSB Structural Database

Known coiled-coil sequences may be retrieved from data banks such as the RCSB protein data bank (http://www.rcsb.org).

### Pentameric Coiled Coils

Pentameric coiled coils can be retrieved from the RCSB database (http://www.rcsb.org/pdb/) by the search for the symmetry in biological assembly using the discriminator “Protein symmetry is cyclic - C5” combined with a text search for “coiled” or “zipper” or combined with a SCOP search like “ScopTree Search for Coiled coil proteins”. A list of suitable entries contains 4PN8 as shown in SEQ ID NO: 40, 4PND as shown in SEQ ID NO: 41, 4WBA as shown in SEQ ID NO: 42, 3V2N as shown in SEQ ID NO: 43, 3V2P as shown in SEQ ID NO: 44, 3V2Q as shown in SEQ ID NO: 45, 3V2R as shown in SEQ ID NO: 46, 4EEB as shown in SEQ ID NO: 47, 4EED as shown in SEQ ID NO: 48, 3MIW as shown in SEQ ID NO: 49, 1MZ9 as shown in SEQ ID NO: 50, 1FBM as shown in SEQ ID NO: 51, 1VDF as shown in SEQ ID NO: 52, 2GUV as shown in SEQ ID NO: 53, 2HYN as shown in SEQ ID NO: 54, 1ZLL as shown in SEQ ID NO: 55, 1T8Z as shown in SEQ ID NO: 56.

### Tetrameric Coiled Coils

Likewise, tetrameric coiled coils can be retrieved using “Protein symmetry is ‘cyclic - C4’” combined with a text search for “coiled” or combined with a SCOP search like “ScopTree Search for Coiled coil proteins”.

For tetrameric coiled coils this yields the following suitable entries: 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2O1K, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5l, 1W5L, 1FE6, 1G1l, 1G1J, 1EZJ, 1RH4, 1GCL.

### Dimeric Coiled Coils

Likewise, dimeric coiled coils can be retrieved using “Protein symmetry is ‘cyclic - C2’” combined with a text search for “coiled” or combined with a SCOP search like “ScopTree Search for Coiled coil proteins”.

For dimeric coiled coils this yields the following suitable entries: 5M97, 5M9E, 5FlY, 5F4Y, 5D3A, 5HMO, 5EYA, 5lX1, 5lX2, 5JHF, 5JVM, 5JVP, 5JVR, 5JVS, 5JVU, 5JX1, 5FCN, 5HHE, 2N9B, 4ZRY, 4Z6Y, 4YTO, 4Zl3, 5AJS, 5F3K, 5F5R, 5HUZ, 5DJN, 5DJO, 5CHX, 5CJ0, 5CJ1, 5CJ4, 5C9N, 5CFF, 4WHV, 3WUT, 3WUU, 3WUV, 4ZQA, 4XA3, 4XA4, 4PXJ, 4YVC, 4YVE, 5BML, 5AL7, 4WOT, 4CG4, 5AMO, 4Wll, 4WIK, 4RSJ, 4CFG, 4R3Q, 4WID, 4CKG, 4CKH, 4NSW, 4W7P, 4QQ4, 4OJK, 4TL1, 4OH9, 4LPZ, 4Q62, 4L2W, 4M3L, 4CKM, 4CKN, 4N6J, 4LTB, 4LRZ, 2MAJ, 2MAK, 4NAD, 4HW0, 4BT8, 4BT9, 4BTA, 4HHD, 4M8M, 4J3N, 4L6Q, 4C1A, 4C1B, 4GDO, 4BWK, 4BWP, 4BWX, 4HU5, 4HU6, 4L9U, 4G0U, 4G0V, 4G0W, 4L3l, 4G79, 4GEU, 4GEX, 4GFA, 4GFC, 4BL6, 4JMR, 4JNH, 2YMY, 4HAN, 3VMY, 3VMZ, 3VN0, 4ABX, 3W03, 2LW9, 4DZM, 4ETO, 3TNU, 3THF, 4E8U, 3VMX, 4E61, 3VEM, 3VBB, 4DJG, 3TV7, 3STQ, 3V8S, 3Q8T, 3U1C, 3QH9, 3AZD, 3ONX, 3OKQ, 3QX3, 3SJA, 3SJB, 3SJC, 2L2L, 3QFL, 3QKT, 2XV5, 2Y3W, 3Q0X, 3AJW, 3NCZ, 3NI0, 2XU6, 3M91, 3NMD, 3LLL, 3LX7, 3ME9, 3MEU, 3MEV, 3ABH, 3ACO, 3IAO, 3HLS, 2WMM, 3A6M, 3A7O, 2WVR, 3ICX, 3ID5, 3ID6, 3HNW, 3I1G, 2K6S, 3GHG, 3G1E, 2W6A, 2V51, 3ERR, 3E1R, 2VY2, 2ZR2, 2ZR3, 3CL3, 3D9V, 2Z17, 2JEE, 3BBP, 3BAS, 3BAT, 2QM4, 2V71, 2NO2, 2PON, 2V0O, 2DQ0, 2DQ3, 2Q2F, 2NRN, 2E7S, 2H9V, 2FXM, 2HJD, 2GZD, 2GZH, 2FV4, 2F2U, 2EUL, 2ESM, 2ETK, 2ETR, 1ZXA, 1YIB, 1YIG, 1XSX, 1RFY, 1U0I, 1XJA, 1T3J, 1T6F, 1R7J, 1UII, 1PL5, 1S1C, 1P9I, 1R48, 1URU, 1OV9, 1UIX, 1NO4, 1NYH, 1MV4, 1LR1, 1L8D, 1LJ2, 1KQL, 1GXK, 1GXL, 1GK6, 1JR5, 1GMJ, 1JAD, 1JCH, 1JBG, 1JTH, 1JY2, 1JY3, 1IC2, 1HCI, 1HF9, 1HBW, 1FXK, 1D7M, 1QUU, 1CE9, 2A93, 1BM9, 1A93, 1TMZ, 2AAC, 1ZII, 1ZIK, 1ZIL, 2ARA, 2ARC, 1JUN, 1YSA, 2ZTA. However, this list of dimeric structures also contains antiparallel coiled coils since dimeric coiled coils with cyclic two-fold symmetry selects parallel and antiparallel coiled-coil. Visual inspection of the structure can easily tell apart the parallel from the antiparallel dimeric coiled coils.

Some of those entries for pentameric, tetrameric and dimeric coiled coils also contain additional protein domains, but upon visual inspection those additional domains can easily be detected and removed.

As an alternative the website http://coiledcoils.chm.bris.ac.uk/ccplus/search/periodic table/ gives a periodic table of coiled-coil structures from which dimeric, trimeric, tetrameric and pentameric (such as 2GUV) coiled coils, but also more complex coiled-coil assemblies such as six-helix bundles (such as 2EBO) can be chosen.

Amino acid modifications of the pentameric, tetrameric and dimeric coiled coil domains used herein are also envisaged. Such modifications may be e.g. the substitution of amino acids that are non-core residues (aa(a) and aa(d)) at the outside of the oligomer at positions aa(e), aa(g), aa(b), aa(c) or aa(f), preferably at positions aa(b), aa(c) or aa(f), most preferably in position aa(f). Possible modifications are substitutions to charged residues to make these oligomers more soluble. Also, shorter constructs of these domains are envisaged.

Other amino acid modifications may be e.g. the substitution of amino acids at core positions (aa(a) and aa(d)) for the purpose of stabilizing the oligomer, i.e. by replacing less favorable core residues by more favorable residues, i.e. as a general rule, residues at core positions with a lower coiled-coil propensity according to Table 1 can be replaced with residues with higher coiled-coil propensity if they do not change the oligomerization state of the coiled coil.

The term “amino acid modification” used herein includes an amino acid substitution, insertion, and/or deletion in a polypeptide sequence, and is preferably an amino acid substitution. By “amino acid substitution” or “substitution” herein is meant the replacement of an amino acid at a particular position in a parent polypeptide sequence with another amino acid. For example, a substitution R94K refers to a variant polypeptide, in which the arginine at position 94 is replaced with a lysine. For the purposes herein, multiple substitutions are typically separated by a slash. Usually 1 to 15, preferably 1 to 10, more preferably 1 to 5, even more preferably 1 to 4, in particular 1 to 3, more particular 1 to 2, most particular 1 amino acid is substituted. For example, R94K/L78V refers to a double variant comprising the substitutions R94K and L78V. By “amino acid insertion” or “insertion” as used herein is meant the addition of an amino acid at a particular position in a parent polypeptide sequence. For example, insert -94 designates an insertion at position 94. By “amino acid deletion” or “deletion” as used herein is meant the removal of an amino acid at a particular position in a parent polypeptide sequence. For example, R94- designates the deletion of arginine at position 94.

A peptide or protein containing an amino acid modification as described herein will preferably possess at least about 80%, most preferably at least about 90%, more preferably at least about 95%, in particular 99% amino acid sequence identity with a parent (un-modified) peptide or protein. Preferably, the amino acid modification is a conservative modification.

As used herein, the term “conservative modification” or “conservative sequence modification” is intended to refer to amino acid modifications that do not significantly alter the biophysical properties of the amino acid sequence. Modifications can be introduced into a protein of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

In one embodiment the oligomerization domain ND1 and/or ND2, preferably ND1 and ND2, is a coiled-coil domain. In a preferred embodiment the oligomerization domain ND1 and/or ND2, preferably ND1 and ND2, is a dimeric, a tetrameric or a pentameric domain, more preferably a tetrameric or a pentameric domain. In a more preferred embodiment the oligomerization domain ND1 and/or ND2, preferably ND1 and ND2, is a pentameric coiled coil selected from the group consisting 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, 1T8Z or a pentameric coiled coil selected from the group consisting of pdb-entries 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, 1T8Z, which contains an amino acid modification and/or is shortened at either or both ends wherein each pentameric coiled coil is indicated according to the pdb entry numbering of the RCSB Protein Data Bank (RCSB PDB). In a further more preferred embodiment the oligomerization domain ND1 and/or ND2, preferably ND1 and ND2, is a pentameric coiled coil selected from the group consisting 4PN8 as shown in SEQ ID NO: 40, 4PND as shown in SEQ ID NO: 41, 4WBA as shown in SEQ ID NO: 42, 3V2N as shown in SEQ ID NO: 43, 3V2P as shown in SEQ ID NO: 44, 3V2Q as shown in SEQ ID NO: 45, 3V2R as shown in SEQ ID NO: 46, 4EEB as shown in SEQ ID NO: 47, 4EED as shown in SEQ ID NO: 48, 3MIW as shown in SEQ ID NO: 49, 1MZ9 as shown in SEQ ID NO: 50, 1FBM as shown in SEQ ID NO: 51, 1VDF as shown in SEQ ID NO: 52, 2GUV as shown in SEQ ID NO: 53, 2HYN as shown in SEQ ID NO: 54, 1ZLL as shown in SEQ ID NO: 55, 1T8Z as shown in SEQ ID NO: 56 or a pentameric coiled coil selected from the group consisting of pdb-entries 4PN8 as shown in SEQ ID NO: 40, 4PND as shown in SEQ ID NO: 41, 4WBA as shown in SEQ ID NO: 42, 3V2N as shown in SEQ ID NO: 43, 3V2P as shown in SEQ ID NO: 44, 3V2Q as shown in SEQ ID NO: 45, 3V2R as shown in SEQ ID NO: 46, 4EEB as shown in SEQ ID NO: 47, 4EED as shown in SEQ ID NO: 48, 3MIW as shown in SEQ ID NO: 49, 1MZ9 as shown in SEQ ID NO: 50, 1FBM as shown in SEQ ID NO: 51, 1VDF as shown in SEQ ID NO: 52, 2GUV as shown in SEQ ID NO: 53, 2HYN as shown in SEQ ID NO: 54, 1ZLL as shown in SEQ ID NO: 55, 1T8Z as shown in SEQ ID NO: 56, which contains an amino acid modification and/or is shortened at either or both ends wherein each pentameric coiled coil is indicated according to the pdb entry numbering of the RCSB Protein Data Bank (RCSB PDB). Even more preferred ND1 and/or ND2, preferably ND1 and ND2, is a pentameric coiled coil selected from the group consisting of the tryptophan-zipper pentamerization domain (pdb-entry: 1T8Z) or a tryptophan-zipper pentamerization domain (pdb-entry: 1T8Z) which contains an amino acid modification and/or is shortened at either or both ends, in particular a pentameric coiled coil comprising SEQ ID NO:3, SEQ ID NO:8 or SEQ ID NO:26). Even more further preferred ND1 and/or ND2, preferably ND1 and ND2, is a pentameric coiled coil selected from the group consisting of the tryptophan-zipper pentamerization domain (pdb-entry: 1T8Z as shown in SEQ ID NO: 56) or a tryptophan-zipper pentamerization domain (pdb-entry: 1T8Z as shown in SEQ ID NO: 56) which contains an amino acid modification and/or is shortened at either or both ends, in particular a pentameric coiled coil comprising SEQ ID NO:3, SEQ ID NO:8 or SEQ ID NO:26). In another more preferred embodiment the oligomerization domain ND1 and/or ND2, preferably ND1 and ND2, is a tetrameric coiled coil selected from the group consisting of 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2O1K, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5l, 1W5L, 1FE6, 1G1I, 1G1J, 1EZJ, 1RH4, 1GCL or a tetrameric coiled coil selected from the group consisting of pdb-entries 5D60, 5D5Y, 5AL6, 4WB4, 4BHV, 4C5Q, 4GJW, 4H7R, 4H8F, 4BXT, 4LTO, 4LTP, 4LTQ, 4LTR, 3ZDO, 3RQA, 3R4A, 3R4H, 3TSI, 3K4T, 3F6N, 2O6N, 2OVC, 2O1J, 2O1K, 2AG3, 2CCE, 1YBK, 1U9F, 1U9G, 1U9H, 1USD, 1USE, 1UNT, 1UNU, 1UNV, 1UNW, 1UNX, 1UNY, 1UNZ, 1UO0, 1UO1, 1UO2, 1UO3, 1UO4, 1UO5, 1W5l, 1W5L, 1FE6, 1G1I, 1G1J, 1EZJ, 1RH4, 1GCL, which contains an amino acid modification and/or is shortened at either or both ends, wherein each tetrameric coiled coil is indicated according to the pdb entry numbering of the RCSB Protein Data Bank (RCSB PDB).

In another more preferred embodiment the oligomerization domain ND1 and/or ND2, preferably ND1 and ND2, is selected from the group of coiled coils comprising SEQ ID NO: 3, SEQ ID NO: 19 and SEQ ID NO: 23.

In a most preferred embodiment the tetrameric coiled coil is from tetrabrachion, preferably the tetrameric coiled coil from tetrabrachion (1FE6) or from tetrabrachion (1FE6) which contains an amino acid modification and/or is shortened at either or both ends, wherein each the tetrabrachion is indicated according to the pdb entry numbering of the RCSB Protein Data Bank (RCSB PDB), in particular the tetrameric coiled coil is a tetrameric coiled coil comprising SEQ ID NO: 19.

In a further most preferred embodiment the tetrameric coiled coil is from tetrabrachion, preferably the tetrameric coiled coil from tetrabrachion (1FE6 as shown in SEQ ID NO: 57) or from tetrabrachion (1FE6 as shown in SEQ ID NO: 57) which contains an amino acid modification and/or is shortened at either or both ends, wherein each the tetrabrachion is indicated according to the pdb entry numbering of the RCSB Protein Data Bank (RCSB PDB), in particular the tetrameric coiled coil is a tetrameric coiled coil comprising SEQ ID NO: 19.

### Specific Coiled Coils

Most preferred are the coiled-coil sequences and monomeric building blocks described in the examples.

### SHBs

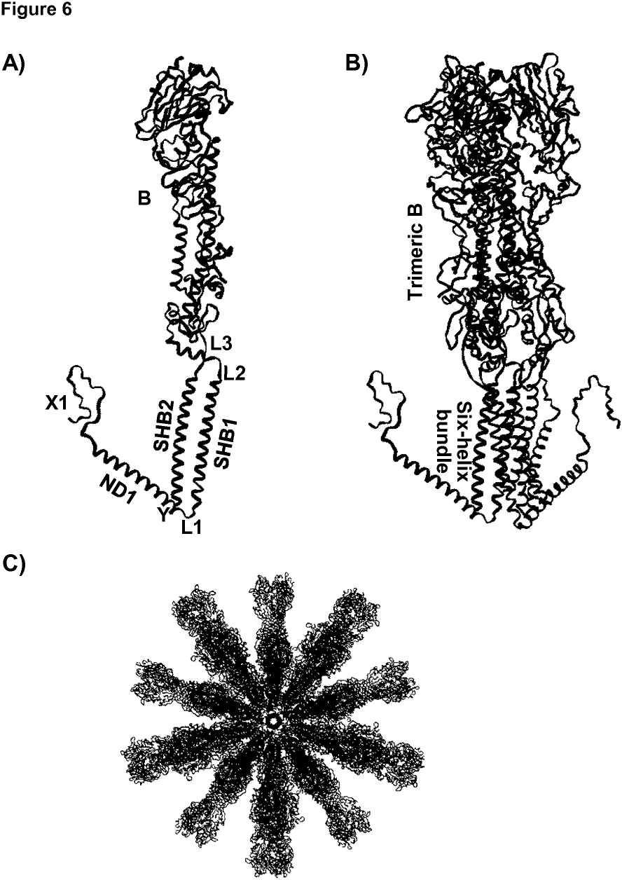

A SHB peptide or protein as used herein refers to a peptide or protein which forms bundles which consist of six helices usually packed in a central trimeric coiled-coil arrangement. A SHB helix as used herein refers to a peptide or protein which is normally a helix which together with five other SHB helices forms a six-helix bundle. A SHB helix is usually an alpha helix. Usually the domains SHB1 and SHB2 of one monomeric building block according to the invention form a six-helix bundle together with the domains SHB1 and SHB2 of two further monomeric building blocks according to the invention as displayed e.g in FIGS. 2B), 6B) and 14B).

SHBs as used herein are usually coiled-coil proteins. SHB-proteins are normally composed of a central trimeric coiled-coil domain that assembles with three other helices that run antiparallel to the central trimeric coiled-coil domain to form a SHB. Connecting the coiled-coil helix with the antiparallel helix by an amino acid sequence therefore generates a loop structure of this sequence upon formation of the SHB. Since the oligomerization state of an SHB is a trimer, trimeric loop-forming proteins can thus be stabilized in their native conformation by using them to connect the two helices of the SHB (FIG. 1).

Coiled-coil SHBs can be retrieved from the RCSB database (http://www.rcsb.org/pdb/) by the search for the stoichiometry in biological assembly using the discriminator “Stoichiometry is A3B3” combined with a text search for “bundle” if the two helices are on separate chains. Suitable entries that contain SHBs are 4I2L, 3W19, 3VTQ, 3VU5, 3VU6, 3VTP, 3VGY, 3VH7, 3VGX, 3VIE, 3RRR, 3RRT, 3KPE, 3G7A, 3F4Y, 3F50, 1ZV8 representing SHBs from HIV, RSV, SARS and paramyxovirus. If the two helices are part of the same protein chain, then stoichiometry “A3” or symmetry is ‘cyclic - C3’ has to be chosen. Combined with the text search for “bundle” and “six” yields the list of the following suitable pdb-entries: 4NJL, 4NSM, 4JF3, 4JGS, 4JPR, 2OT5, 3CP1, 3CYO, 2IEQ, 1JPX, 1JQ0, 1K33, 1K34.

A de novo design of SHB proteins has also been described (Boyken, S. E., et al. Science 2016, 352(6286): 680-687). The pdb-entries for these structures are 5J0J, 5J0I, 5J0H, 5IZS, 5J73, 5J2L, 5J0L, 5J0K, 5J10. Amino acid modifications of the SHBs used herein are also envisaged. Such modifications may be e.g. the substitution of amino acids that are non-core residues (aa(a) and aa(d)) at the outside of the core trimer at positions aa(e), aa(g), aa(b), aa(c) or aa(f), preferably at positions aa(b), aa(c) or aa(f), most preferably in position aa(f). Other residues are the surface exposed residues of the antiparallel helix. However, these modifications may not interfere with the ability of the SHB1 to form a six-helix bundle complex with SHB2. Possible modifications are substitutions to charged residues to make the SHB more soluble. Also shorter constructs of these domains are comprised by the present invention. Shorter constructs of these domains usually comprise at least three heptad-repeats (i.e. at least 21 amino acids) in the central coiled-coil domain, without being bound by theory, the interaction of SHB1 with SHB2 usually needs at least six helix turns - corresponding to three heptad repeats of the central trimeric coiled coil - to be specific enough. More preferably, the central coiled-coil domain is at least four heptad repeats long. Other modifications may be e.g. the substitution of amino acids at core positions (aa(a) and aa(d)) for the purpose of stabilizing the core trimer, i.e. by replacing less favorable residues by more favorable residues, i.e. as a general rule, residues at core positions with a lower coiled-coil propensity according to Table 1 can be replaced with residues with higher coiled-coil propensity if they do not change the oligomerization state of the coiled coil. In Example 5) the modification T560V replaces a threonine at an aa(d) position with a valine, thus replacing threonine with a coiled-coil propensity of -1.2 by valine with a higher propensity of 1.1 at the core position aa(d). Likewise, T564V replaces a threonine at an aa(a) position with a valine, thus replacing threonine with a coiled-coil propensity of 0.2 by valine with a much higher propensity of 4.1 at the core position aa(a).

In a preferred embodiment, the domains SHB1 and/or SHB2 are each independently selected from the group consisting of 4I2L, 3W19, 3VTQ, 3VU5, 3VU6, 3VTP, 3VGY, 3VH7, 3VGX, 3VIE, 3RRR, 3RRT, 3KPE, 3G7A, 3F4Y, 3F50, 1ZV8, 4NJL, 4NSM, 4JF3, 4JGS, 4JPR, 2OT5, 3CP1, 3CYO, 2IEQ, 1JPX, 1JQ0, 1K33, 1K34, 5J0J, 5J0I, 5J0H, 5IZS, 5J73, 5J2L, 5J0L, 5J0K, and 5J10, or independently selected from the group consisting of 4I2L, 3W19, 3VTQ, 3VU5, 3VU6, 3VTP, 3VGY, 3VH7, 3VGX, 3VIE, 3RRR, 3RRT, 3KPE, 3G7A, 3F4Y, 3F50, 1ZV8, 4NJL, 4NSM, 4JF3, 4JGS, 4JPR, 2OT5, 3CP1, 3CYO, 2IEQ, 1JPX, 1JQ0, 1K33, 1K34, 5J0J, 5J0I, 5J0H, 5IZS, 5J73, 5J2L, 5J0L, 5J0K, and 5J10 which contain an amino acid modification and/or is shortened at either or both ends, wherein each SHB is indicated according to the pdb entry numbering of the RCSB Protein Data Bank (RCSB PDB).

In a further preferred embodiment, the domains SHB1 and/or SHB2 are each independently selected from the group consisting of 4I2L as shown in SEQ ID NO: 58, 3W19 as shown in SEQ ID NO: 59, 3VTQ as shown in SEQ ID NO: 60, 3VU5 as shown in SEQ ID NO: 61, 3VU6 as shown in SEQ ID NO: 62, 3VTP as shown in SEQ ID NO: 63, 3VGY as shown in SEQ ID NO: 64, 3VH7 as shown in SEQ ID NO: 65, 3VGX as shown in SEQ ID NO: 66, 3VIE as shown in SEQ ID NO: 67, 3RRR as shown in SEQ ID NO: 68, 3RRT as shown in SEQ ID NO: 69, 3KPE as shown in SEQ ID NO: 70, 3G7A as shown in SEQ ID NO: 71, 3F4Y as shown in SEQ ID NO: 72, 3F50 as shown in SEQ ID NO: 73, 1ZV8 as shown in SEQ ID NO: 74, 4NJL as shown in SEQ ID NO: 75, 4NSM as shown in SEQ ID NO: 76, 4JF3 as shown in SEQ ID NO: 77, 4JGS as shown in SEQ ID NO: 78, 4JPR as shown in SEQ ID NO: 79, 2OT5 as shown in SEQ ID NO: 80, 3CP1 as shown in SEQ ID NO: 81, 3CYO as shown in SEQ ID NO: 82, 2IEQ as shown in SEQ ID NO: 83, 1JPX as shown in SEQ ID NO: 84, 1JQ0 as shown in SEQ ID NO: 85, 1K33 as shown in SEQ ID NO: 86, 1K34 as shown in SEQ ID NO: 87, 5J0J as shown in SEQ ID NO: 88, 5J0I as shown in SEQ ID NO: 89, 5J0H as shown in SEQ ID NO: 90, 5IZS as shown in SEQ ID NO: 91, 5J73 as shown in SEQ ID NO: 92, 5J2L as shown in SEQ ID NO: 93, 5J0L as shown in SEQ ID NO: 94, 5J0K as shown in SEQ ID NO: 95, and 5J10 as shown in SEQ ID NO: 96, or independently selected from the group consisting of 4I2L as shown in SEQ ID NO: 58, 3W19 as shown in SEQ ID NO: 59, 3VTQ as shown in SEQ ID NO: 60, 3VU5 as shown in SEQ ID NO: 61, 3VU6 as shown in SEQ ID NO: 62, 3VTP as shown in SEQ ID NO: 63, 3VGY as shown in SEQ ID NO: 64, 3VH7 as shown in SEQ ID NO: 65, 3VGX as shown in SEQ ID NO: 66, 3VIE as shown in SEQ ID NO: 67, 3RRR as shown in SEQ ID NO: 68, 3RRT as shown in SEQ ID NO: 69, 3KPE as shown in SEQ ID NO: 70, 3G7A as shown in SEQ ID NO: 71, 3F4Y as shown in SEQ ID NO: 72, 3F50 as shown in SEQ ID NO: 73, 1ZV8 as shown in SEQ ID NO: 74, 4NJL as shown in SEQ ID NO: 75, 4NSM as shown in SEQ ID NO: 76, 4JF3 as shown in SEQ ID NO: 77, 4JGS as shown in SEQ ID NO: 78, 4JPR as shown in SEQ ID NO: 79, 2OT5 as shown in SEQ ID NO: 80, 3CP1 as shown in SEQ ID NO: 81, 3CYO as shown in SEQ ID NO: 82, 2IEQ as shown in SEQ ID NO: 83, 1JPX as shown in SEQ ID NO: 84, 1JQ0 as shown in SEQ ID NO: 85, 1K33 as shown in SEQ ID NO: 86, 1K34 as shown in SEQ ID NO: 87, 5J0J as shown in SEQ ID NO: 88, 5J0I as shown in SEQ ID NO: 89, 5J0H as shown in SEQ ID NO: 90, 5IZS as shown in SEQ ID NO: 91, 5J73 as shown in SEQ ID NO: 92, 5J2L as shown in SEQ ID NO: 93, 5J0L as shown in SEQ ID NO: 94, 5J0K as shown in SEQ ID NO: 95, and 5J10 as shown in SEQ ID NO: 96, which contain an amino acid modification and/or is shortened at either or both ends, wherein each SHB is indicated according to the pdb entry numbering of the RCSB Protein Data Bank (RCSB PDB).

In a more preferred embodiment SHB1 and/or SHB2 is a peptide selected from the group consisting SEQ ID NO:5, SEQ ID NO:7, SEQ ID NO:17, SEQ ID NO:19, SEQ ID NO:28, SEQ ID NO:30, SEQ ID NO:32, SEQ ID NO:33, SEQ ID NO:34 and SEQ ID NO:35.

### Domain B

The domain B is a peptide or protein comprising a loop region. Usually, the domain B is a peptide or protein comprising a loop region wherein the domain comprises an antigen. Antigens to be comprised by domain B of the present invention can be either B-cell epitopes and/or T-cell epitopes and are selected from the group consisting of (a) proteins or peptides which induce an immune response against cancer cells; (b) proteins, peptides or carbohydrates which induce an immune response against infectious diseases; (c) proteins or peptides which induce an immune response against allergens; and (d) protein or peptide hormones which induce an immune response for the treatment of a human disease. SAPNs comprising such proteins, or peptidic fragments thereof may be suited to induce an immune response in humans, or also in farm animals and pets. Particular useful antigens comprised by domain B are a protein or peptide which induces an immune response against cancer cells, a protein or peptide which induces an immune response against infectious diseases, protein or peptide which induces an immune response against allergens, protein or peptide which induces an immune response for the treatment of a human disease.

Most preferably, antigens to be comprised by domain B of the present invention and to be displayed in a loop-conformation on the SAPNs are selected from the group consisting of trimeric surface glycoproteins of enveloped viruses. There are many different classification schemes for viruses. Typically, viral fusogens belong to one of three different classes (Podbilewicz, B. Annu Rev Cell Dev Biol. 2014, 30: 111-139). The class of special interest is Class I, a well-known member of which is influenza with its surface protein HA. This Class I includes fusogens from a variety of different viral families such as paramyxoviruses, filoviruses, retroviruses, and coronaviruses, to name a few. The structural feature of interest of class I fusogens are triple-helical prefusion glycoproteins, which rearrange into a six-helix bundle to form the so-called the postfusion conformation. The most important viral species of interest with their trimeric surface glycoprotein include influenza virus A and B (HA - see Example 5), HIV (gp160 - see Example 12), Ebola (GP), Marburg (GP), RSV (F-protein), CMV (gB protein - see Example 1), HSV (gB protein), SARS (S-protein) and MERS (S-protein). Also fragments of these surface glycoproteins can be displayed in trimeric oligomerization state as loop-forming proteins (see Example 1 and Example 12).

Of particular interest are loop-structured proteins that form trimers such as many of the surface proteins of enveloped viruses, which display such a trimeric loop structure. Examples are the influenza HA, the gB protein of CMV, the F protein of RSV, the gp160 of HIV and many more. These trimeric surface proteins of enveloped viruses are in a metastable pre-fusogenic state that can be stabilized by engineering it on the helix-loop-helix motif of the SHB of the nanoparticles of the present invention. Alternatively, substructures of trimeric proteins can be held together in trimeric conformation using the SHB as a scaffold. One particular substructure is shown in Example 12 in form of the V1V2 loop structure of the tip of gp160 of HIV. Also, simple loop structures can be displayed as loops on the SHB without the need and emphasis to form a particular trimeric conformation but simply to be restrained into a loop structure. Thus in a preferred embodiment, the domain B has a trimeric loop structure.

In another preferred embodiment the domain B is selected from a protein or peptide, which induces an immune response against cancer cells, a protein or peptide which induces an immune response against infectious diseases, a protein or peptide which induces an immune response against allergens, a protein or peptide which induces an immune response for the treatment of a human disease. More preferably B is selected from a protein or peptide, which induces an immune response against cancer cells, a protein or peptide which induces an immune response against allergens, a protein or peptide which induces an immune response for the treatment of a human disease, in particular B is selected from a protein or peptide, which induces an immune response against cancer cells and/or a protein or peptide which induces an immune response against allergens.

In another preferred embodiment the domain B is selected from the group of trimeric surface glycoproteins of enveloped viruses of Class I.

In another preferred embodiment the domain B is selected from the group consisting of trimeric surface glycoproteins of influenza virus A and B (HA), HIV (gp160), Ebola (GP), Marburg (GP), RSV (F-protein), CMV (gB protein), HSV (gB protein), SARS (S-protein) and MERS (S-protein). In another preferred embodiment the domain B is selected from the group consisting of influenza HA, the gB protein of CMV, the F protein of RSV, the gp160 of HIV and the protein with pdb entry 4TVP or selected from the group consisting of influenza HA, the gB protein of CMV, the F protein of RSV, the gp160 of HIV and the protein with pdb code 4TVP which contains an amino acid modification and/or is shortened at either or both ends. Particularly, preferably the domain B is selected from the group consisting of influenza HA, the gB protein of CMV, the gp160 of HIV and the protein with pdb entry 4TVP or selected from the group consisting of influenza HA, the gB protein of CMV, the gp160 of HIV and the protein with pdb code 4TVP which contains an amino acid modification and/or is shortened at either or both ends (Example 12). In another preferred embodiment the domain B is selected from the group consisting of a protein comprising SEQ ID NO:6, SEQ ID NO:18 and SEQ ID NO:29.

The loop region is usually a protein in which the N-terminal end and the C-terminal end of the particular loop are in close proximity such that they can be engineered onto the two helices of the SHB, which are also in close proximity. Depending on the particular amino acid positions of the two helices to which the loop structure is attached by means of the linker L2 and L3, the distance between the attachment points varies to some degree. For the six-helix bundle from RSV (pdb-code 5J3D) the shorter distances between Cα-positions of the peptide chains is about 5 Å (at the helix-helix interface) while the longer distances are about 15 Å (at opposite sides of the helices). For the six-helix bundle from HIV (pdb-code 3G7A) the distances between Cα-positions of the peptide chains are very comparable with values between 5.5 Å to about 15 Å for the shorter and longer distances, respectively. Adding the length of the linkers L2 and L3 to the longest distance gives the maximum distance that both ends of B can be apart from each other. For HA the distance between the N-terminal and C-terminal end in the crystal structure of pdb-code 3SM5 is 15.8 Å (Examples 5 to 9), while for the V1V2 loop of Example 12 the distance between the N-terminal and C-terminal end in the crystal structure of pdb-code 4TVP is 13.1 Å. In a preferred embodiment the loop region is usually a protein in which the distance between the N-terminal and C-terminal end in the crystal structure is between about 3 Å and about 20 Å, preferably between about 5 Å and about 17 Å.

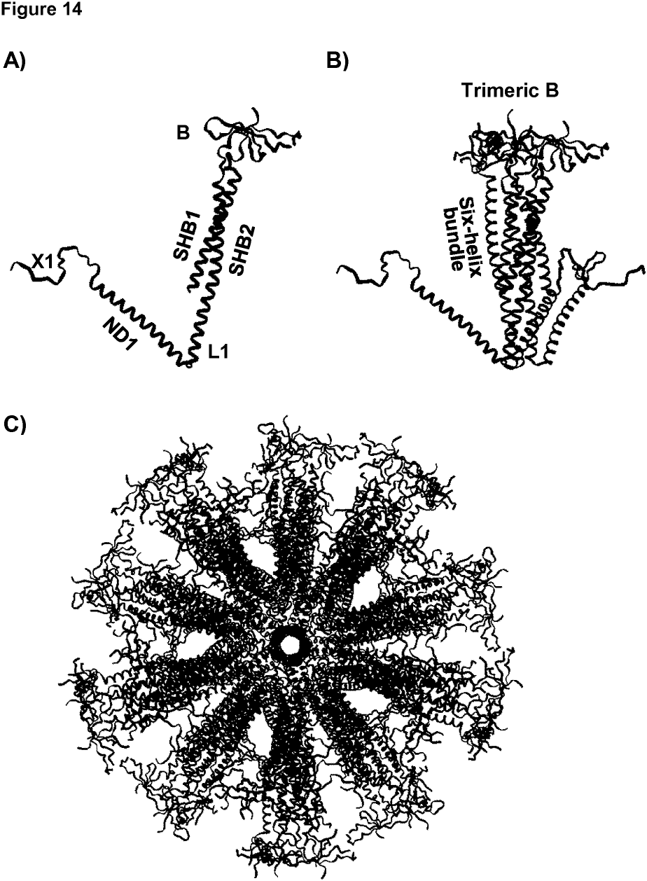

In a preferred embodiment either the N-terminal or the C-terminal end of B are in α-helical conformation such that B can be attached to SHB1 or SHB2 by means of a continuous α-helix such as for the V1V2 loop of gp160 in Example 12 (FIG. 14). If the domain B is a simple β-turn, then the distance between the N- and C-terminal ends is about 4.5 Å. A typical β-turn structure that can be used as domain B is the V3 loop of HIV gp160. The distance between possible N-terminal and C-terminal ends in the crystal structure of pdb-code 4TVP is 4.6 Å (residues 306 to 318), 6.7 Å (residues 300 to 326) or 4.2 Å (residues 296 to 331) for the V3 loop of HIV gp160. In a preferred embodiment the domain B is a simple β-turn and the distance between possible N-terminal and C-terminal ends is between about 3 Å and about 8 Å, preferably between about 4 Å and about 7 Å.

### Linkers

A linker chain L1, L2 or L3 is composed of either a single peptide bond or a peptide chain, preferably, a peptide chain consisting of 1 to 50 amino acids or a single peptide bond, more preferably a peptide chain consisting of 1 to 30 amino acids or a single peptide bond, even more preferably a peptide chain consisting of 1 to 20 amino acids or a single peptide bond, most preferably a peptide chain consisting of 1 to 15 amino acids or a single peptide bond.

In a preferred embodiment, the linker chain L1, L2 or L3 is selected from the group consisting of a peptide bond, AAA, GS, GG, SEQ ID NO:4, SEQ ID NO:13, SEQ ID NO:15, SEQ ID NO:20, and SEQ ID NO:27. Preferably, the linker L1 contains an α-helical segment connecting to the SHB1 domain, more preferably contains a coiled-coil sequence in register with the following SHB1 domain. If the SHB1 domain is the central trimeric coiled coil of the SHB this α-helical segment of L1 is preferably part of a coiled-coil sequence. For example, in the sequence L1 of Example 1 the portion ELYSRLAEIE (SEQ ID NO:36) is a coiled coil in register with the coiled coil of following SHB1 domain. Likewise, residues 1 to 8 of L1 of Example 5 represent a coiled-coil stretch in register with the preceding SHB1 domain. Again, residues 4 to 14 of L1 in Example 12 contain a coiled-coil sequence in register with the following SHB1 domain.

### Self-Assembling Protein Nanoparticles: LCM Units

SAPNs are formed from monomeric building blocks of formula (la) or (lb) and/or formula (lla) or (IIb). If such building blocks assemble, they will form so-called “LCM units”. The number of monomeric building blocks, which will assemble into such an LCM unit will be defined by the least common multiple (LCM). Hence, if for example the oligomerization domains of the monomeric building block form a pentamer (ND1)₅ (m=5) and a trimeric SHB, 15 monomers will form an LCM unit. If the linker segment L2 has the appropriate length, this LCM unit may assemble in the form of a spherical protein nanoparticle. SAPNs may be formed by the assembly of only one or more than one LCM units (Table 2). Such SAPNs represent topologically closed structures.

### Regular Polyhedra

There exist five regular polyhedra, the tetrahedron, the cube, the octahedron, the dodecahedron and the icosahedron. They have different internal rotational symmetry elements. The tetrahedron has a 2-fold and two 3-fold axes, the cube and the octahedron have a 2-fold, a 3-fold and a 4-fold rotational symmetry axis, and the dodecahedron and the icosahedron have a 2-fold, a 3-fold and a 5-fold rotational symmetry axis. In the cube the spatial orientation of these axes is exactly the same as in the octahedron, and also in the dodecahedron and the icosahedron the spatial orientation of these axes relative to each other is exactly the same. Hence, for the purpose of SAPNs of the invention the dodecahedron and the icosahedron can be considered to be identical. The dodecahedron / icosahedron is built up from 60 identical three-dimensional building blocks (Table 2). These building blocks are the asymmetric units (AUs) of the polyhedron. They are pyramids and the pyramid edges correspond to one of the rotational symmetry axes, hence these AUs will carry at their edges 2-fold, 3-fold, and 5-fold symmetry elements. If these symmetry elements are generated from protein oligomerization domains such AUs are constructed from monomeric building blocks as described above. It is sufficient to align the two oligomerization domains ND1 and/or ND2, preferably ND1 and ND2, and SHB½ along two of the symmetry axes of the AU. The SHB formed by SHB1 and SHB2 has always trimeric symmetry. ND1 and/or ND2, preferably ND1 and ND2, may be a pentamer, tetramer or dimer. If these two oligomerization domains form stable oligomers, the symmetry interface along the third symmetry axis will be generated automatically, and it may be stabilized by optimizing interactions along this interface, e.g. hydrophobic, hydrophilic or ionic interactions, or covalent bonds such as disulfide bridges.

### Assembly to Self-Assembling Protein Nanoparticles (SAPNs) With Regular Polyhedral Symmetry

To generate self-assembling protein nanoparticles (SAPNs) with a regular geometry (dodecahedron, icosahedron, octahedron, cube and tetrahedron), more than one LCM unit is needed. E.g. to form an icosahedron from a monomer containing trimeric and pentameric oligomerization domains, 4 LCM units, each composed of 15 monomeric building blocks are needed, i.e. the protein nanoparticle with regular geometry will be composed of 60 monomeric building blocks. The combinations of the oligomerization states of the two oligomerization domains needed and the number of LCM units to form the corresponding polyhedra are listed in Table 2.

TABLE 2

Possible combinations of oligomerization states in the formation of regular polyhedra

| ID No. | m | Polyhedron Type | LCM | No. of LCM Units | No. of Building Blocks |
|---|---|---|---|---|---|
| 1 | 5 | dodecahedron / icosahedron | 15 | 4 | 60 |
| 2 | 4 | cube / octahedron | 12 | 2 | 24 |
| 3 | 2 | tetrahedron | 6 | 2 | 12 |
| 4 | 2 | cube / octahedron | 6 | 4 | 24 |
| 5 | 2 | dodecahedron / icosahedron | 6 | 10 | 60 |

Whether the LCM units will further assemble to form regular polyhedra composed of more than one LCM unit depends on the geometrical alignment of the two oligomerizations domains ND1 and/or ND2, preferably ND1 and ND2, and SHB½ with respect to each other, especially on the angle between the rotational symmetry axes of the two oligomerization domains. This is mainly governed by i) the interactions between neighboring domains in a nanoparticle, ii) the length of the linker segment L2, iii) the shape of the individual oligomerization domains. This angle is larger in the LCM units compared to the arrangement in a regular polyhedron. Also this angle is not identical in monomeric building blocks as opposed to the regular polyhedron.

If the angle between the two oligomerization domains is sufficiently small (even smaller than in a regular polyhedron with icosahedral symmetry), then a large number (several hundred) protein chains can assemble into a protein nanoparticle. A biophysical and mathematical analysis of SAPNs with trimer-pentamer architecture has recently been published (Indelicato, G., et al. Biophys J 2016, 110(3): 646-660).

In a further aspect, the invention relates to monomeric building blocks of formula (la) or (lb) or formula (lla) or (llb)as defined above.

In another aspect, the invention relates to composition comprising a protein nanoparticle as herein described. Such a composition is particularly suitable as a vaccine. Preferred vaccine compositions comprise the protein nanoparticle in an aqueous buffer solution, and may further comprise, for example, sugar derived excipients (such as glycerol, trehalose, sucrose, etc.) or amino acid derived excipients (such as arginine, proline, glutamate, etc.) or anionic, cationic, non-ionic or twitter-ionic detergents (such as cholate, deoxycholate, tween, etc.) or any kind of salt (such as NaCl, MgCl₂, etc.) to adjust the ionic strength of the solution.

In another aspect, the invention relates to a method of vaccinating a human or non-human animal, which comprises administering an effective amount of a protein nanoparticle as described hereinbefore to a subject in need of such vaccination.

The invention also relates to a protein nanoparticle as described hereinbefore for use in a method of vaccinating a human or non-human animal, which comprises administering an effective amount of a protein nanoparticle as described hereinbefore to a subject in need of such vaccination.

The invention also relates to the use of a protein nanoparticle as described hereinbefore for the manufacture of a medicament for vaccinating a human or non-human animal, which comprises administering an effective amount of a protein nanoparticle as described hereinbefore to a subject in need of such vaccination.

### DESIGN OF AN SHB-SAPN (SELF-ASSEMBLING PROTEIN NANOPARTICLE WITH THE SHB)

A particular example of an SHB-SAPN according to the invention is the following construct “HC_AD1g”, corresponding to formula (la) with the sequence

MGHHHHHHKRGSWREWNAKWDEWENDWNDWREDWQAWRDDWAYWTLTWRY
GELYSRLAEIETLLRGIVQQQQQLLDVVKRQQEMLRLVVWGTKNLQARVA
EAWCVDQRRTLEVFKELSKINPSAILSAIYNKPIAARFMGDVLGLASCVT
INQTSVKVLRDMNVKESPGRCYSRPVVIFNFARSEYVQYGQLGEDNEILL
GNHRTEECQLPSLKIFIAGNSAYEYVDYLFKRMIDDGGEGPYRVCSMAQG
TDLIRFERNIVCTGTDEDKQEWEHKIRFLEANISESLEQAQIQQEKNMYE
LQKL (SEQ IDNO:1)

This is a construct composed of the following partial structures:

| | |
|---|---|
| X1: | MGHHHHHHKRGS (SEQ ID NO:2) |
| ND1: | WREWNAKWDEWENDWNDWREDWQAWRDDWAYWTLTW (SEQ ID NO:3) |
| L1: | RYGELYSRLAEIE (SEQ ID NO:4) |

| | |
|---|---|
| SHB1: | TLLRGIVQQQQQLLDVVKRQQEMLRLVVWGTKNLQARV (SEQ ID NO:5) |
| L2: | peptide bond |
| B: | AEAWCVDQRRTLEVFKELSKINPSAILSAIYNKPIAARFMGDVLGLASCVTINQTS VKVLRDMNVKESPGRCYSRPVVIFNFARSEYVQYGQLGEDNEILLGNHRTEECQL PSLKIFIAGNSAYEYVDYLFKRMIDDGGEGPYRVCSMAQGTDLIRFERNIVCT (SEQ ID NO:6) |
| L3: | GTDEDK (SEQ ID NO:15) |
| SHB2: | QEWEHKIRFLEANISESLEQAQIQQEKNMYELQKL (SEQ ID NO:7) |
| Y1: | absent |

For ease of purification HC_AD1g starts with the sequence X1 as defined in formula (la) or (Ib):

MGHHHHHHKRGS (SEQ ID NO:2)

which contains a His-tag for nickel affinity purification and at the DNA level restriction sites for further sub-cloning (Ncol and BamHI).

For ND1 a pentamerization domain was chosen (m=5). The particular pentameric coiled coil is a novel modification of the tryptophan-zipper pentamerization domain (Liu, J., et al. Proc Natl Acad Sci USA 2004, 101(46): 16156-16161) with pdb-entry 1T8Z.

The original tryptophan-zipper pentamerization domain has the sequence

SSNAKWDQWSSDWQTWNAKWDQWSNDWNAWRSDWQAWKDDWARWNQRWDN
WAT (SEQ IDNO: 8)

The modified coiled-coil sequence of the pentamerization domain used for HC_AD1g starts at position 13, ends at position 49 and contains sequence variations at the C-terminal end (TLTW instead of NQRW) and for solubility purposes several charge modifications at non-core positions of the coiled-coil but keeping the heptad repeat pattern of the tryptophane residues at core positions as in the original sequence (SEQ ID NO:8).

13-WREWNAKWDEWENDWNDWREDWQAWRDDWAYWTLTW-48 (SEQ ID
NO:3)

This sequence is extended then by the short linker L1 RYGELYSRLAEIE (SEQ ID NO:4), then connected with the first helix of the SHB SHB1 from gp41 of HIV. L1 contains a flexible residue G (glycine) between the pentamer and the trimer parts of the nanoparticle followed by the coiled-coil stretch ELYSRLAEIE (SEQ ID NO:36) leading into the SHB of HIV with the following sequence:

TLLRGIVQQQQQLLDVVKRQQEMLRLVVWGTKNLQARV (SEQ ID NO:
5)

This SHB1 sequence corresponds to residues 534 to 571 of the HIV gp41 protein P12449.1 with the sequence

534-TLFRGIVQQQQQLLDVVKRQQEMLRLTVWGTKNLQARV-571 (SE
Q ID NO:9)

with the two point mutations F536L and T560V wherein the two point mutations F536L and T560V further stabilize the core coiled-coil trimer of the SHB. The two helices of the SHB within the envelope glycoprotein of HIV (P12449.1) has the following sequence (in bold):

MSGKIQLLVAFLLTSACLIYCTKYVTVFYGVPVWKNASIPLFCATKNRDT
WGTIQCLPDNDDYQEIPLNVTEAFDAWDNIVTEQAVEDVWNLFETSIKPC
VKLTPLCVTMNCNASTESAVATTSPSGPDMINDTDPCIQLNNCSGLREED
MVECQFNMTGLELDKKKQYSETWYSKDVVCESDNSTDRKRCYMNHCNTSV
ITESCDKHYWDAMRFRYCAPPGFVLLRCNDTNYSGFEPNCSKVVASTCTR
MMETQPSTWLGFNGTRAENRTYIYWHGRDNRTIISLNKYYNLTILCRRPE
NKTVVPITLMSGRRFHSQKIINKKPRQAWCRFKGEWREAMQEVKQTLVKH
PRYKGTNDTNKINFTAPEKDSDPEVAYMWTNCRGEFLYCNMTWFLNWVEN
KTGQQHNYVPCHIEQIINTWHKVGKNVYLPPREGELSCESTVTSIIANID
VDGDNRTNITFSAEVAELYRLELGDYKLVEVTPIGFAPTAEKRYSSAPGR
HKRGVLVLGFLGFLTTAGAAMGAASLTLSAQSRTLFRGIVQQQQQLLDVV KRQQEMLRLTVWGTKNLQARVTAIEKYLADQARLNSWGCAFRQVCHTTVP
WVNDTLTPEWNNMTWQEWEHKIRFLEANISESLEQAQIQQEKNMYELQKL
NSWDVFGNWFDLTSWIKY IQYGVMIVVGIVALRIVIYVVQMLSRLRKGY
RPVFSSPPGYIQQIHIHKDWEQPDREETEEDVGNDVGSRSWPWPIEYIHF
LIRLLIRLLTRLYNSCRDLLSRLYLILQPLRDWLRLKAAYLQYGCEWIQE
AFQALARVTRETLTSAGRSLWGALGRIGRGILAVPRRIRQGAEIALL (S
EQ ID NO:10)

This SHB1 is then followed by a peptide bond to the next amino acid alanine of the loop-forming protein B with the sequence:

AEAWCVDQRRTLEVFKELSKINPSAILSAIYNKPIAARFMGDVLGLASCV
TINQTSVKVLRDMNVKESPGRCYSRPVVIFNFARSEYVQYGQLGEDNEIL
LGNHRTEECQLPSLKIFIAGNSAYEYVDYLFKRMIDDGGEGPYRVCSMAQ
GTDLIRFERNIVCT (SEQ ID NO:6)

This loop-forming protein B is somewhat more complex. It contains the tip of the gB protein of CMV with the AD1 domain. The residues 504 to 638

(AEAWCVDQRRTLEVFKELSKINPSAILSAIYNKPIAARFMGDVLGLASC
VTINQTSVKVLRDMNVKESPGRCYSRPVVIFNFANSSYVQ YGQLGEDNE
ILLGNHRTEECQLPSLKIFIAGNSAYEYVDYLFKRMID (SEQ ID NO:
11))

are linked to residues 90 to 112 (PYRVCSMAQGTDLIRFERNIVCT (SEQ ID NO:12) by the peptide string DGGEG (SEQ ID NO:13). This generates a continuous loop-forming protein domain of the tip region of the gB protein (FIG. 2A) that then is held together by the SHB to a trimeric conformation (FIG. 2B). It also contains two point mutations N587R and S589E to make it more soluble. The sequence of the full-length gB protein is:

MESRIWCLVVCVNLCIVCLGAAVSSSSTRGTSATHSHHSSHTTSAAHSRS
GSVSQRVTSSQTVSHGVNETIYNTTLKYGDVVGVNTTKYPYRVCSMAQGT DLIRFERNIVCTSMKPINEDLDEGIMVVYKRNIVAHTFKVRVYQKVLTFR
RSYAYIHTTYLLGSNTEYVAPPMWEIHHINSHSQCYSSYSRVIAGTVFVA
YHRDSYENKTMQLMPDDYSNTHSTRYVTVKDQWHSRGSTWLYRETCNLNC
MVTITTARSKYPYHFFATSTGDVVDISPFYNGTNRNASYFGENADKFFIF
PNYTIVSDFGRPNSALETHRLVAFLERADSVISWDIQDEKNVTCQLTFWE
ASERTIRSEAEDSYHFSSAKMTATFLSKKQEVNMSDSALDCVRDEAINKL
QQIFNTSYNQTYEKYGNVSVFETTGGLVVFWQGIKQKSLVELERLANRSS
LNLTHNRTKR|STDGNNATHLSNMESVHNLVYAQLQFTYDTLRGYINRAL
AQIAEAWCVDQRRTLEVFKELSKINPSAILSAIYNKPIAARFMGDVLGLA SCVTINQTSVKVLRDMNVKESPGRCYSRPVVIFNFANSSYVQYGQLGEDN EILLGNHRTEECQLPSLKIFIAGNSAYEYVDYLFKRMIDLSSISTVDSMI
ALDIDPLENTDFRVLELYSQKELRSSNVFDLEEIMREFNSYKQRVKYVED
KVVDPLPPYLKGLDDLMSGLGAAGKAVGVAIGAVGGAVASVVEGVATFLK
NPFGAFTIILVAIAVVIIIYLIYTRQRRLCMQPLQNLFPYLVSADGTTVT
SGNTKDTSLQAPPSYEESVYNSGRKGPGPPSSDASTAAPPYTNEQAYQML
LALVRLDAEQRAQQNGTDSLDGQTGTQDKGQKPNLLDRLRHRKNGYRHLK
DSDEENV (SEQ ID NO:14)

This B domain is then followed the peptide linker L3 with the sequence GTDEDK (SEQ ID NO:15) to the connected with the second helix of the SHB SHB2 from gp41 of HIV of the following sequence:

QEWEHKIRFLEANISESLEQAQIQQEKNMYELQKL (SEQ ID NO:7)

This corresponds to residues 616 to 650 of the HIV gp41 protein P12449.1 (SEQ ID NO:10). Finally, the fragment Y1 of formula (la) is absent in this construct HC_AD1g.

A model of HC_AD1g monomer is shown in FIG. 2 in its monomeric, trimeric and icosahedral forms, assuming T=1 icosahedral symmetry. An EM picture of HC_AD1g is shown in FIG. 3.

### EXAMPLES

The following examples are useful to further explain the invention but in no way limit the scope of the invention.

### Example 1 - Cloning

The DNA coding for the nanoparticle constructs were prepared using standard molecular biology procedures. For example, the plasmids containing the DNA coding for the protein sequence HC_AD1g

MGHHHHHHKRGSWREWNAKWDEWENDWNDWREDWQAWRDDWAYWTLTWRY
GELYSRLAEIETLLRGIVQQQQQLLDVVKRQQEMLRLVVWGTKNLQARVA
EAWCVDQRRTLEVFKELSKINPSAILSAIYNKPIAARFMGDVLGLASCVT
INQTSVKVLRDMNVKESPGRCYSRPVVIFNFARSEYVQYGQLGEDNEILL
GNHRTEECQLPSLKIFIAGNSAYEYVDYLFKRMIDDGGEGPYRVCSMAQG
TDLIRFERNIVCTGTDEDKQEWEHKIRFLEANISESLEQAQIQQEKNMYE
LQKL (SEQID NO:1)

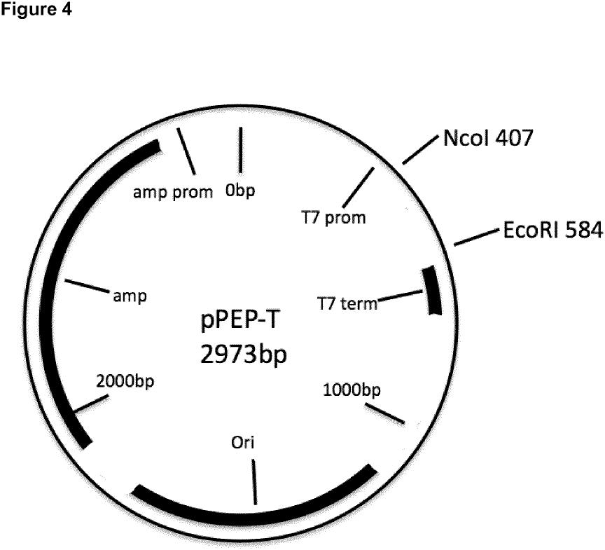

was constructed by cloning into the Ncol/EcoRI restriction sites of the basic SAPN expression construct of FIG. 4.

This construct with the formula (la) X1 - ND1 - L1 - SHB1 - L2 - B - L3 - SHB2 - Y1 is composed of a His-tag (X1), a pentameric coiled-coil tryptophane zipper (ND1) a linker (L1) the trimeric coiled-coil of gp41 of the HIV SHB (SHB1) a peptide bond as linker (L2), the tip of the glycoprotein gB of CMV (B) forming a trimeric loop structure (B) a linker (L3) connecting the C-terminus of B to the second helix of the SHB within the gp41 of HIV (SHB2), while Y1 in this construct is absent.

### Example 2 - Expression

The plasmids were transformed into Escherichia coli BL21 (DE3) cells, which were grown in Luria broth with ampicillin at 37° C. Other cell lines as tuner BL21 (DE3), Origami 2(DE3) and Rosetta 2(DE3)pLysS can be used. Expression was induced with isopropyl β-D-thiogalactopyranoside. Four hours after induction, cells were removed from 37° C. and harvested by centrifugation at 4,000 x g for 15 min. The cell pellet was stored at -20° C. The pellet was thawed on ice and suspended in a lysis buffer consisting of 9 M urea, 100 mM NaH₂PO₄, 10 mM Tris pH 8, 20 mM imidazole, and 0.2 mM Tris-2-carboxyethyl phosphine (TCEP).

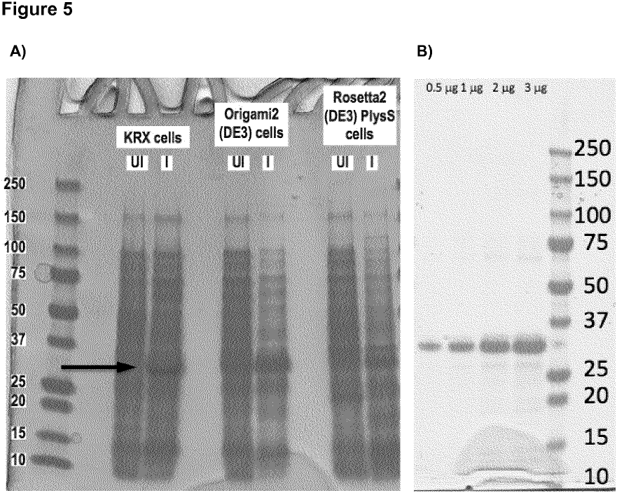

Alternatively, also other cell lines can be used for expression, such as KRX cells. In KRX cells expression can be done with the early auto-induction protocol of KRX cells using O/N pre-culture at 37 degree with Amp (100 µg/mL) and glucose (0.4%). Diluting the O/N pre-cultures 1:100 into the expression culture containing Amp (100 µg/mL), glucose (0.05%) and rhamnose (0.1%) at 25° C. for 24 hours. The protein expression level was assessed by sodium dodecyl sulfate polyacrylamide gel electrophoresis (SDS-PAGE; FIG. 5A).

### Example 3 - Purification

Cells were lysed by sonication and the lysate was cleared by centrifuging at 30,500 x g for 45 min. The cleared lysate was incubated with Ni-NTA Agarose Beads (Qiagen, Valencia, CA, USA) for at least 1 hour. The column was washed with lysis buffer and then the purified with the following wash and elution protocol:
Lysis Buffer: 100 mM NaH₂PO₄, 10 mM Tris, 9 M Urea, 5 mM DTT, pH 8.0
Wash 1: Lysis Buffer
Wash 2: 500 mM NaH₂PO₄, 10 mM Tris, 9 M Urea, 5 mM DTT, pH 8.0
Wash 3: 100 mM NaH₂PO₄, 20 mM Citric Acid, 9 M Urea, 5 mM DTT, pH 6.3
Wash 4: 100 mM NaH₂PO₄, 20 mM Citric Acid, 9 M Urea, 5 mM DTT, pH 5.9
Wash 5: 100 mM NaH₂PO₄, 20 mM Citric Acid, 9 M Urea, 5 mM DTT, pH 4.5
Wash 6: Lysis Buffer
Wash 7: 60% isopropanol, 10 mM Tris, pH 8.0 (removal of Endotoxin)
Wash 8: Lysis Buffer
Wash 9: Lysis Buffer
Elution: Lysis Buffer with 250 mM Imidazole
Purity was assessed by sodium dodecyl sulfate polyacrylamide gel electrophoresis (SDS-PAGE) as shown in FIG. 5B.

### Example 4 - Refolding

For refolding the protein was rebuffered to the following conditions: pH 8.5, 20 mM Tris, 50 mM NaCl, 5% Glycerol, 1 mM TCEP. For quick refolding 6.7 mL protein (16.75 mg) was refolded in 328 mL of refolding buffer composed of pH 8.0, 20 mM Tris, 50 mM NaCl, 5% Glycerol. The final protein concentration after refolding was 0.05 mg/mL. After quick refolding the protein was dialyzed 2× 4000 L in the refolding buffer to remove the remaining urea. The solution was then analyzed by negative stain transmission electron microscopy at different resolutions. EM pictures of HC-AD1g after refolding show nice nanoparticle formation (FIG. 3).

### Example 5 - Architecture of the Influenza Vaccine F34-HAPR-HIVlong

On the computer graphics an influenza HA-based SHB-SAPN coined “F34-HAPR-HIVlong” with the following sequence has been designed:

MGNNMTWQEWEHKIRFLEANISESLEQAQIQQEKNMYELQKLNSWDVFGA
AADADTICIGYHANNSTDTVDTVLEKNVTVTHSVNLLEDSHNGKLCRLKG
IAPLQLGKCNIAGWLLGNPECDPLLPVRSWSYIVETPNSENGICYPGDFI
DYEELREQLSSVSSFERFEIFPKESSWPNHNTNGVTAACSHEGKSSFYRN
LLWLTEKEGSYPKLKNSYVNKKGKEVLVLWGIHHPPNSKEQQNLYQNENA
YVSVVTSNYNRRFTPEIAERPKVRDQAGRMNYYWTLLKPGDTIIFEANGN
LIAPMYAFALSRGFGSGIITSNASMHECNTKCQTPLGAINSSLPYQNIHP
VTIGECPKYVRSAKLRMVTGLRNIPSIQSRGLFGAIAGFIEGGWTGMIDG
WYGYHHQNEQGSGYAADQKSTQNAINGITNKVNTVIEKMNIQFTAVGKEF
NKLEKRMENLNKKVDDGFLDIWTYNAELLVLLENERTLDFHDSNVKNLYE
KVKSQLKNNAKEIGNGCFEFYHKCDNECMESVRNGTYDYPKYSEESKGST
LSAQVRTLLAGIVQQQQQLLDVVKRQQEMLRLVVWGVKNLQARVTAIEKY
LKRLRAALQGGAIINETADDIVYRLTVIIDDRYESLKNLITLRADRLEMI
INDNVSTILASIGGDEGDEGDEAREGHHHHHHHHHHGS (SEQ ID NO:
16)

F34-HAPR-HIVlong is a construct that has an architecture according to formula (lb) and is composed of the following partial structures:
Y1: MGSHB2: NNMTWQEWEHKIRFLEANISESLEQAQIQQEKNMYELQKLNSWDVFG (SEQ ID NO:17)
L3:AAAB:DADTICIGYHANNSTDTVDTVLEKNVTVTHSVNLLEDSHNGKLCRLKGIAPLQLGKCN IAGWLLGNPECDPLLPVRSWSYIVETPNSENGICYPGDFIDYEELREQLSSVSSFERFEIFPK ESSWPNHNTNGVTAACSHEGKSSFYRNLLWLTEKEGSYPKLKNSYVNKKGKEVLVLWGIH HPPNSKEQQNLYQNENAYVSVVTSNYNRRFTPEIAERPKVRDQAGRMNYYWTLLKPGDTII FEANGNLIAPMYAFALSRGFGSGIITSNASMHECNTKCQTPLGAINSSLPYQNIHPVTIGECP KYVRSAKLRMVTGLRNIPSIQSRGLFGAIAGFIEGGWTGMIDGWYGYHHQNEQGSGYAAD QKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPK YSEESK (SEQ ID NO:18)
L2:GSSHB1:TLSAQVRTLLAGIVQQQQQLLDVVKRQQEMLRLVVWGVKNLQARVTAIEKYL (SEQ ID NO:19)
L1:KRLRAALQGGA (SEQ ID NO:20)
ND1:IINETADDIVYRLTVIIDDRYESLKNLITLRADRLEMIINDNVSTILASI (SEQ ID NO:21)
X1:GGDEGDEGDEAREGHHHHHHHHHHGS (SEQ ID NO:22)

The particular origin and function of the sections of this influenza vaccine construct are the as follows. Y1 contains at the DNA level the cloning site for Ncol; SHB2 is a long form (residues 611 to 657) of the gp41 SHB of the HIV sequence P12449.1; L3 contains the restrictions site for Notl; B corresponds to the residues 16 to 511 of the HA protein P03452.2 of influenza A virus A/Puerto Rico/8/1934(H1N1); L2 contains the restriction site for BamHI; SHB1 is a long form (residues 527 to 578) of the other helix of the gp41 SHB of the HIV sequence P12449.1 with four point mutations to stabilize the coiled-coil trimer (F536L, R537A, T560V and T564V); L1 contains a short coiled-coil stretch, the restriction site for Pstl and the flexible GG sequence between the trimer and the tetramer coiled coil; ND1 contains residues 3 to 52 of the sequence from the crystal structure of tetrabrachion with pdb-code 1YBK forming a tetrameric coiled coil; X1 contains a stretch of charged residues followed by the His-Tag.

### Example 6 - Cloning

The sequence encoding F34-HAPR-HIVlong was ordered with flanking restriction sites (Ncol/EcoRI) from Genscript. Ncol and EcoRI restriction enzymes were used to subclone F34-HAPR-HIVlong into the pPEP-T expression vector (FIG. 4).

### Example 7 - Protein Expression, Purification and Refolding

The F34-HAPR-HIVlong constructs were transformed into BL21(DE3) expression cells (New England BioLabs) and expressed in Hyper Broth Medium (Athena). Freshly transformed bacteria colony was used to inoculated 10 mL Hyper Broth with ampicillin (100 ug/mL) and grown overnight at 28° C. (200 rpm). 1% of the overnight culture was used to inoculate the expression culture (Hyper Broth with ampicillin, 100 ug/mL). The expression culture was grown at 37° C., 200 rpm. Culture was induced for 3 h at 37° C. using IPTG (final concentration of 1 mM) when cell density at OD600 nm reached 0.8. Cell pellet was collected by centrifugation (4000 g, 4° C.) and washed with ice-cold 1xPBS. Purification was performed under denaturing and reducing condition. Cell pellet was resuspended in the lysis buffer (pH 8.0, 8 M Urea, 10 mM Tris, 100 mM NaH₂PO₄, 2 mM TCEP) and sonicated for 3 min (40% amplitude, 3 sec puls on 3 sec puls off) followed by centrifugation (14′000xg, 50 min, 4° C.) to pellet cell debris. The proteins were purified using a 5 mL HisTrap column (GE Healthcare) on a AKTA Prime FPLC (GE Healthcare). Protein binding was performed at a flow rate of 0.5 mL/min followed by wash 1 (Lysis Buffer, flow rate 2 mL/min), wash 2 (Lysis Buffer containing 10 mM Imidazole, pH 8.0), wash 3 (pH 8, 8 M Urea, 10 mM Tris, 500 mM NaH₂PO₄, 10 mM Imidazole, 2 mM TCEP), wash 4 (pH 4.5, 8 M Urea, 20 mM Sodium Citrate, 100 mM

NaH₂PO₄, 10 mM Imidazole, 2 mM TCEP), wash 5 (pH 8.0, 10 mM Tris, 60% isopropanol) followed by equilibrating back to wash buffer 2 before elution. Protein was eluted with elution buffer (pH 8.0, 8 M Urea, 10 mM Tris, 100 mM NaH₂PO₄, 2 mM TCEP, 500 mM Imidazole). Protein containing fraction were pooled and incubated with EDTA 5 mM final concentration to chelate released Nickel (incubation 1 h at RT) and rebuffered to the pre-refolding buffer (6 M GndHCl, 50 mM Tris, 100 mM NaCl, 10 mM EDTA, 10 mM TCEP, 10% Glycerol, pH 8.0). Protein concentration was measured by OD280 reading. Refolding was performed by a 100-fold dilution adding the protein drop-wise (4× 1 mL in a 90 min interval) to the refolding buffer (100 mM Tris, 400 mM L-Arginine, 2 mM EDTA, 5 mM GSH, 1 mM GSSG, 25% Glycerol, pH 8.0) under constant stirring. Refolded particles were filtered (0.1 um PES membrane filter, Sartolab, Satorius) and concentrated with Amicon Ultra (100 kDa cut off, Millipore) and filtered (0.1 um syringe filter, Minisart, Sartorius) again. Particle preparation showed a final concentration of 0.37 mg/mL. Throughout the refolding, filtration, concentration and final filtration process protein loss was 65%.

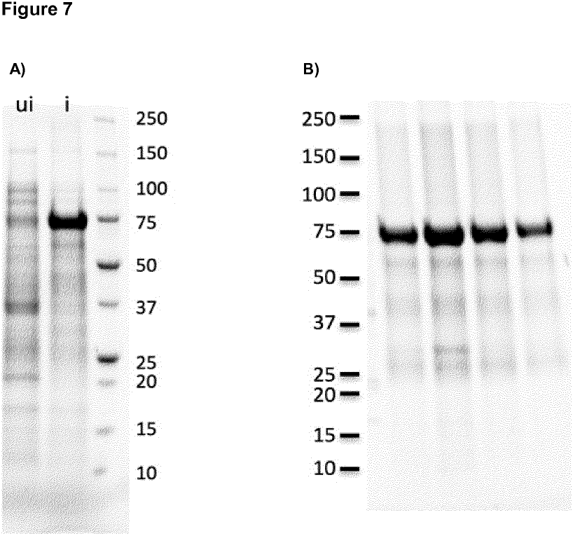

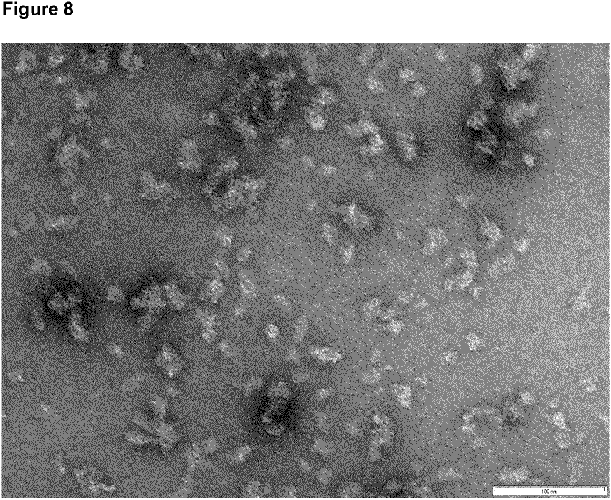

SDS-PAGE analysis of the expression culture showed nice expression of the F34-HAPR-HIVlong monomer running at the predicted molecular weight of 77.9 kDa (FIG. 7A). The protein is expressed in inclusion bodies (data not shown) and could be affinity purified with high purity after solubilization in denaturing buffer condition (FIG. 7B) and formed nanoparticles as evidenced by electron microscopy (FIG. 8).

### Example 8 - F34-HAPR-HIVlong Characterization Using mAB Directed Against the Globular Head and Polyclonal HA-Specific Hyperimmune Sera

Correct refolding of HA on the SHB-SAPNs was verified by an ELISA binding assay with either a conformation-specific monoclonal antibody (IC5-4F8, BEI Resources) or a polyclonal hyperimmune serum (NIBSC) in comparison with an inactivated influenza PR8/34 virus. Plates were coated in triplicates with either refolded F34-HAPR-HIVlong particles (1.7 µg/mL) or inactivated virus PR8/34 (1.7 µg/mL) in coating buffer (pH 9.0, 100 mM NaHCO₃, 12 mM

Na₂CO₃) overnight at 4° C. As negative control only coating buffer was added in 3 wells. Plates were washed 3x with wash buffer (1x DPBS, 0.05% Tween, 300 uL/well) and blocked with blocking buffer (1x DPBS, 3% BSA, 300 µL/well) for 2 h at RT on a shaker. The commercial monoclonal Anti-Influenza A virus HA, clone IC5-4F8 (1:500; BEI Resources) that was shown to recognize the correctly folded trimeric globular head on the virus was used to analyze the globular head formation on the surface of our particles. To further characterize the refolded HA molecule on the surface of the particle the commercial available Influenza anti A/Puerto Rico/8/34 (H1N1) polyclonal hyperimmune sheep sera (1:1000, NIBSC) was used. Plates were washed 3x with wash buffer (300 µL/well) and the secondary antibody, anti-mouse-lgG peroxidase labeled (1:5000 in 1xPBS/3%BSA, 100 µL/well, Sigma) or anti goat/sheep-IgG peroxidase labeled (1:1000, in 1xPBS/3%BSA, 100 µL/well, Sigma) respectively was added and incubated for 1 h at RT. Plates were washed 3x with washing buffer and developed by the addition of TMB developing solution (100 µL/well, Sigma). Reaction was stopped after 15 min or 2 min respectively using 0.5 M sulfuric acid (100 µL/well), color reaction was read using the ELISA reader (Tecan GENios Pro) at 450 nm.

Since the inactivated virus is fixed in formalin we can expect the HA molecules at the surface of the inactivated virus to show the correct conformation. A strong recognition of the F34-HAPR-HIVlong particles by both the conformation-specific mAb IC5-4F8 and the polyclonal immune serum was observed, confirming correct folding of HA on the SHB-SAPNs. The recognition was only somewhat reduced compared to the inactivated virus by both sera suggesting that a fraction of the HA molecules on the SHB-SAPNs are not correctly folded (FIGS. 9A,B). For the globular head specific mAb we see a reduction of 1.6-fold with the hyperimmune sera a reduction of 1.8-fold compared to the recognition of the inactivated virus.

### Example 9 - Competition ELISA Analysis to Analyze Correct HA Conformation

Incubation of F34-HAPR-HIVlong in coating buffer can demonstrate that HA has the correct conformation to bind antibodies and prevent them from biding to the coated inactivated virus. Therefore, we performed an inhibition ELISA assay to determine if soluble particles compete with antibody recognition of the inactivated virus. ELISA plates were coated with inactivated virus PR8/34 (1 µg/mL) in coating buffer (pH 9.0, 100 mM NaHCO₃, 12 mM Na₂CO₃) overnight at 4° C. Plates were washed 3x with wash buffer (1x DPBS, 0.05% Tween, 300 µL/well) and blocked with blocking buffer (1x DPBS, 3% BSA, 300 µL/well) for 2 h at RT on a shaker. The commercial monoclonal Anti-Influenza A virus HA, clone IC5-4F8 (1:500; BEI Resources) and the commercial available Influenza anti A/Puerto Rico/8/34 (H1N1) hyperimmune polyclonal sheep sera (1:1000, NIBSC) were pre-incubated with 80 ng of F34-HAPR-HIVlong in the particles buffer (pH 8.0, 100 mM Tris, 400 mM L-Arginine, 2 mM EDTA, 5 mM GSH, 1 mM GSSG, 25% Glycerol), for 1 h before adding to the ELISA plates (100 µL/well). As positive control antibody mixture without particle preincubation was analyzed on the same plate. The antibody/particle mixture was incubated for 1 h at RT on the shaker. Plates were washed 3x with wash buffer (300 µL/well) and the secondary antibody, anti-mouse-lgG peroxidase labeled (1:5000 in 1xPBS/3%BSA, 100 µL/well, Sigma) or anti goat/sheep-IgG peroxidase labeled (1:1000, in 1xPBS/3%BSA, 100 µL/well, Sigma) respectively was added and incubated for 1 h at RT. Plates were washed 3x with washing buffer and developed by the addition of TMB developing solution (100 µL/well, Sigma). Reaction was stopped after 15 min or 2 min respectively using 0.5 M sulfuric acid (100 µL/well), color reaction was read using the ELISA reader (Tecan GENios Pro) at 450 nm.

Soluble F34-HAPR-HIVlong could compete with the antibody binding to the inactivated virus PR8/34 (FIGS. 9C,D). 80 ng of F34-HAPR-HIVlong could inhibit the PR8/34 recognition by the mAb by 1.9-fold and by the hyperimmune sera by 4.6-fold. This data confirms that HA on the SAPNs has the right conformation to compete binding of the conformation-specific antibodies to the coated virus.

### Example 10 - F3-HAPR Characterization Using mAB Directed Against the Globular Head and Polyclonal HA-Specific Hyperimmune Sera

A construct similar to F34-HAPR-HIVlong was engineered that lacks the tetramerization domain from tetrabrachion and therefore only forms trimers upon refolding. The HA molecule is stabilized in its pre-fusion trimeric conformation by attachment to the SHB of HIV, but further assembly into SAPNs is not possible since the second oligomerization domain is lacking. This construct is coined F3-HAPR and has the following sequence:

MGNNMTWQEWEHKIRFLEANISESLEQAQIQQEKNMYELQKLNSWDVFGA
AADADTICIGYHANNSTDTVDTVLEKNVTVTHSVNLLEDSHNGKLCRLKG
IAPLQLGKCNIAGWLLGNPECDPLLPVRSWSYIVETPNSENGICYPGDFI
DYEELREQLSSVSSFERFEIFPKESSWPNHNTNGVTAACSHEGKSSFYRN
LLWLTEKEGSYPKLKNSYVNKKGKEVLVLWGIHHPPNSKEQQNLYQNENA
YVSVVTSNYNRRFTPEIAERPKVRDQAGRMNYYWTLLKPGDTIIFEANGN
LIAPMYAFALSRGFGSGIITSNASMHECNTKCQTPLGAINSSLPYQNIHP
VTIGECPKYVRSAKLRMVTGLRNIPSIQSRGLFGAIAGFIEGGWTGMIDG
WYGYHHQNEQGSGYAADQKSTQNAINGITNKVNTVIEKMNIQFTAVGKEF
NKLEKRMENLNKKVDDGFLDIWTYNAELLVLLENERTLDFHDSNVKNLYE
KVKSQLKNNAKEIGNGCFEFYHKCDNECMESVRNGTYDYPKYSEESKGST
LSAQVRTLLAGIVQQQQQLLDVVKRQQEMLRLVVWGVKNLQARVTAIEKY
LKRLRAALQGGGDEGDEGDEAREGHHHHHHHHHHGS (SEQ ID NO:23
)

The construct was cloned, expressed, purified and refolded using the protocol described in Examples 6 and 7 and the subject to the characterization using polyclonal HA-specific hyperimmune serum to probe for correct refolding of the HA molecule on F3-HAPR in comparison to the plates coated with inactivated influenza PR8/34 virus. In particular, refolding was performed by a 100-fold dilution, 2× 500 mL in an interval of 90 min (total 1 mL of protein in 100 mL of refolding buffer of 100 mM Tris, 400 mM L-Arginine, 2 mM EDTA, 5 mM GSH, 1 mM GSSG, pH 8.0 and probing different glycerol concentrations of 5%, 10%, 20% and 20%. The refolded material was concentrated using 30 kDa cut off Amicon concentrator and filtered using 0.2 mm filter to a volume of about 3 mL and protein concentrations of 70 mg/mL, 58 mg/mL, 25 mg/mL and 26 mg/mL for the increasing glycerol concentrations, respectively.

To characterize the refolded HA molecule on the F3-HAPR trimer the commercial available Influenza anti A/Puerto Rico/8/34 (H1N1) polyclonal hyperimmune sheep serum (1:1000, NIBSC) was used. Plates were washed 3x with wash buffer (300 µL/well) and the secondary antibody, anti-mouse-IgG peroxidase labeled (1:5000 in 1xPBS/3%BSA, 100 µL/well, Sigma) or anti goat/sheep-IgG peroxidase labeled (1:1000, in 1xPBS/3%BSA, 100 µL/well, Sigma) respectively was added and incubated for 1 h at RT. Plates were washed 3x with washing buffer and developed by the addition of TMB developing solution (100 µL/well, Sigma). Reaction was stopped after 15 min or 2 min respectively using 0.5 M sulfuric acid (100 µL/well), color reaction was read using the ELISA reader (Tecan GENios Pro) at 450 nm. In FIG. 10 the ELISA shows almost identical profiles for the bacterially expressed F3-HAPR and the inactivated influenza PR8/34 virus for their binding specificities to the polyclonal serum stored at various temperature conditions. This indicates that HA when stabilized by the SHB on F3-HAPR construct is correctly folded even when expressed in a standard BL21(DE3) bacterial expression system.

### Example 11 - Mouse Immunization and Challenge Experiments

Immunization and challenge experiments were performed. Balb/c mice (5 animals per group) were immunized intra muscular (day 0, 14 and 28) with 30 ug of F34-HAPR-HIVlong, inactivated virus PR8/34 (positive control group) or PBS (negative control group). Bleeds were collected (day 14, 28, 41). Mice were challenged with PR8/34 virus on day 42 with a lethal dose of 100 PFU (10 LD90) of A/PR/8/34 (H1N1), the mice were daily monitored (survival, health, weight) until day 14 after challenge.

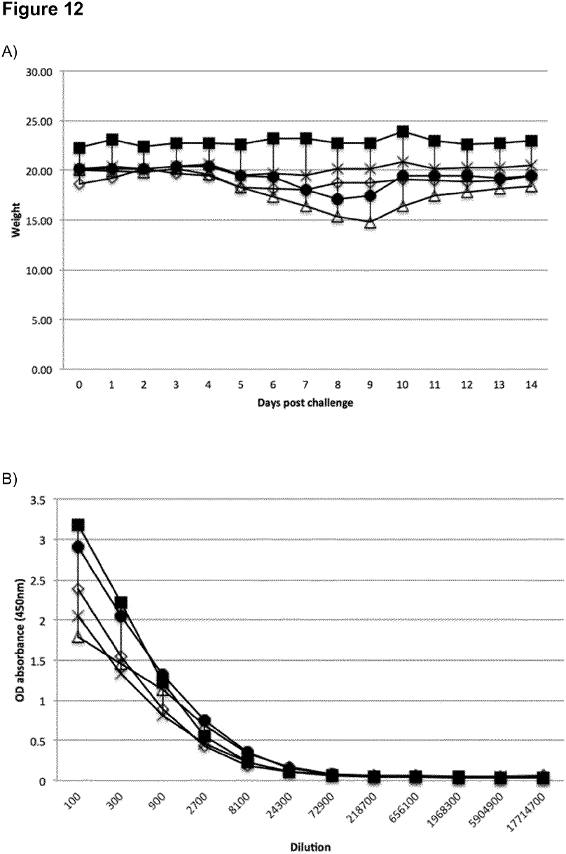

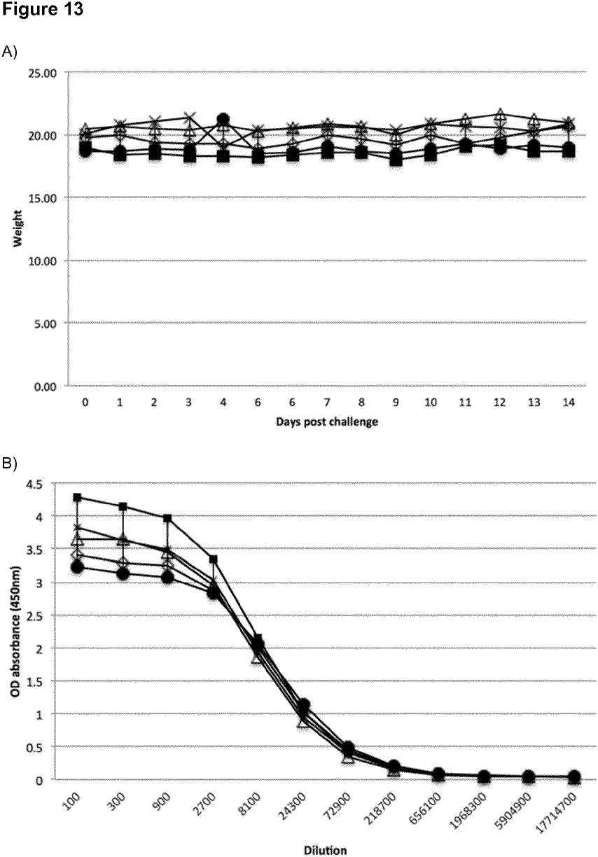

All animals (group of 5 mice) immunized with F34-HAPR-HIVlong survived homologous challenge (FIGS. 11 and 12A). 100% survival was also observed as expected for the group immunized with the inactivated virus PR8/34 (FIGS. 11 and 13A). All control group mice that were immunized with PBS developed severe health status and died (FIG. 11).

The highly protective antibodies induced by F34-HAPR-HIVlong immunization showed only weak recognition of the inactivated virus PR8/34 in the ELISA assay (FIG. 12B), while there were much higher antibody titers specific for the inactivated virus PR8/34 observed in the immunization with the inactivated virus PR8/34 (FIG. 13B).

This indicates that while on the chemically inactivated virus mainly the tip of HA is accessible to the immune system, F34-HAPR-HIVlong presents HA much better as also portions on the side of the HA molecules are surface accessible. Thus, F34-HAPR-HIVlong can induce a wider variety of antibodies than the inactivated virus and therefore potentially be more broadly protective since the tip of HA is highly variable while on the side of the HA molecule the more conserved region of the stem domain is displayed.

### Example 12 - Architecture of the HIV Vaccine 4TVP-1ENV

On the computer graphics an HIV gp160-based SHB-SAPN coined “4TVP-1ENV” with the following sequence has been designed:

MGDKHHHHHHHHHHKDGSDKGSWEEWNARWDEWENDWNDWREDWQAWRDD
WARWRATWMGGRLLSRLERLERRNVEARQLLSGIVQQQNNLLRAIEAQQH
LLQLTVWVKLTPLCVTLQCTNVTNNITDDMRGELKNCSFNMTTELRDKKQ
KVYSLFYRLDVVQINENQGNRSNNSNKEYRLINCNTSAIMEWDREINNYT
SLIHSLIEESQNQQEKNEQELLELDK(SEQ ID NO:24)

4TVP-1ENV is a construct that has an architecture according to formula (la) and is composed of the following partial structures:

| | |
|---|---|
| X1: | MGDKHHHHHHHHHHKDGSDKGS (SEQ ID NO:25) |
| ND1: | WEEWNARWDEWENDWNDWREDWQAWRDDWARWRATW (SEQ ID NO:26) |
| L1: | MGGRLLSRLERLERRNV (SEQ ID NO:27) |
| SHB1: | EARQLLSGIVQQQNNLLRAIEAQQHLLQLTVW (SEQ ID NO:28) |
| L2: | peptide bond |
| B: | VKLTPLCVTLQCTNVTNNITDDMRGELKNCSFNMTTELRDKKQKVYSLFYRLDVVQ INENQGNRSNNSNKEYRLINCNTSAI (SEQ ID NO:29) |
| L3: | peptide bond |
| SHB2: | MEWDREINNYTSLIHSLIEESQNQQEKNEQELLELDK (SEQ ID NO:30) |
| Y1: | absent |

It is based on the crystal structures 4TVP and 1ENV from the RCSB protein database of the proteins gp120 and gp41 of HIV. 4TVP is the crystal structure of the hiv-1 bg505 sosip.664 env trimer ectodomain, comprising the pre-fusion gp120 and gp41, in complex with human antibodies PGT122 and 35O22 (Pancera, M., et al. Nature 2014, 514(7523): 455-461). 1ENV is the atomic structure of the ectodomain from HIV-1 gp41 (Weissenhorn, W., et al. Nature 1997, 387(6631): 426-430), i.e. the SHB.

In particular, it contains in X1 the His-tag as well as the restriction sites for Ncol and BamHI, in ND1 a pentameric coiled-coil tryptophane zipper with many point mutations at non-core residues to make it more soluble. L1 is a linker that contains the flexible GG between pentamer and trimer followed by a coiled-coil sequence. SHB1 contains residues 31 to 61 of chain A from 1ENV. B contains residues 90 to 170 of chain G from 4TVP. SHB2 contains residues 87 to 123 of chain A from 1ENV. Since the V1-V2 loop in B is optimally modelled onto the SHB the linkers L2 and L3 are just peptide bonds. Y1 finally is absent in this construct design.

Since HIV is highly variable, many other combinations of a similar design can be envisaged. In 4TVP the V1V2-loop has long V1 and V2 loops. To focus the immune response to the more conserved portions of gp120, sequences with short V1 and V2 loops can be chosen. Also, to display structures with a lower degree of glycosylation might expose the protein backbone better and induce more broadly neutralizing antibody responses. Therefore, choosing sequences in which some of the glycosylation sites show mutations might be favorable. A possible option would be a combination of the sequences ACZ06517.1, ABW95233.1 and AFU33883.1 to yield a sequence VKLTPLCVTLICKDTTNSTGTMKNCSFS VTTELRDKKQKVYALFYKLDIVPIETGEYRLINCNTSVI (SEQ ID NO:31) for B, in which both loops have short forms and two glycosylation sites are altered to be unglycosylated.

Also, variations of the SHB sequence could be envisaged. The sequences of 1ENV could be replaced by 4TVP (QARNLLSGIVQQQSNLLRAPEAQQHLLKLTVW (SEQ ID NO:32) and LQWDKEISNYTQIIYGLLEESQNQQEKNEQDLLALD (SEQ ID NO:33)) or a more soluble form of the SHB (SEQ ID NO:5 and SEQ ID NO:7)) or the T865/T651 pair (Bai, X., et al. Biochemistry 2008, 47(25): 6662-6670) (QARQLLSGIVQQQNNLLRAIEAQQHLLQLTVW (SEQ ID NO:34) and MEWDREINNYTSLIHSLIEESQNQQEKNEQELLELDK (SEQ ID NO:35)), which is almost identical to 1ENV. Shorter forms of these helices will also work as long as the helices still form a stable enough SHB (see reference Bai, X., et al. Biochemistry 2008, 47(25): 6662-6670).

1. A self-assembling protein nanoparticle (SAPN) consisting of a multitude of building blocks of formula (Ia) or (Ib) 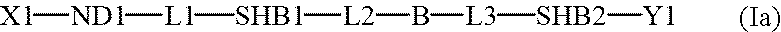 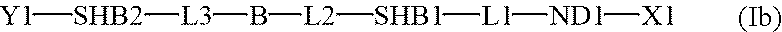
consisting of a continuous chain comprising an oligomerization domain ND1, a linker L1, a domain SHB1, a linker L2, a domain B comprising a loop region, a linker L3, a domain SHB2, and further substituents X1 and Y1, wherein
ND1 is a peptide or protein that comprises oligomers (ND1)ₘ of m subunits ND1,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X1 is absent or a peptide or protein sequence comprising 1 to 1000 amino acids that may be further substituted,
Y1 is absent or a peptide or protein sequence comprising 1 to 1000 amino acids that may be further substituted,
wherein the multitude of building blocks of formula (Ia) or formula (Ib) is optionally co-assembled with a multitude of building blocks of formula (IIa) or formula (IIb) 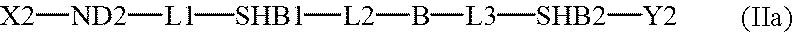 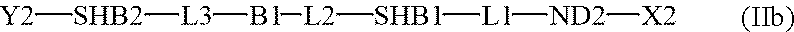
consisting of a continuous chain comprising an oligomerization domain ND2, a linker L1, a domain SHB1, a linker L2, a domain B comprising a loop region, a linker L3, a domain SHB2, and further substituents X2 and Y2, wherein
ND2 is a peptide or protein that comprises oligomers (ND2)ₘ of m subunits ND2,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X2 is absent or a peptide or protein sequence comprising 1 to 1000 amino acids that may be further substituted,
Y2 is absent or a peptide or protein sequence comprising 1 to 1000 amino acids that may be further substituted,
and wherein at least one of X2 and Y2 of formula (IIa) and/or formula (IIb) is different from X1 and Y1 of formula (Ia) and/or formula (Ib).

2. The protein nanoparticle according to claim 1 wherein the oligomerization domain ND1, the linker L1, the domain SHB1, the linker L2, the domain B comprising a loop region, the linker L3, and the domain SHB2 of formula (Ia) or formula (Ib) are identical to the oligomerization domain ND2, the linker L1, the domain SHB1, the linker L2, the domain B comprising a loop region, the linker L3, and the domain SHB2 of formula (IIa) or formula (IIb).

3. The protein nanoparticle according to claim 1 wherein ND1 and/or ND2 is a coiled-coil.

4. The protein nanoparticle according to claim 3 wherein ND1 and/or ND2 is a pentameric coiled coil.

5. The protein nanoparticle according to claim 4 wherein ND1 and/or ND2 is a pentameric coiled coil selected from the group consisting of 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, and 1T8Z or wherein ND1 and/or ND2 is a pentameric coiled coil selected from the group consisting of 4PN8, 4PND, 4WBA, 3V2N, 3V2P, 3V2Q, 3V2R, 4EEB, 4EED, 3MIW, 1MZ9, 1FBM, 1VDF, 2GUV, 2HYN, 1ZLL, and 1T8Z which contains an amino acid modification and/or is shortened at either or both ends, wherein each coiled coil is indicated according to the pdb entry numbering of the RCSB Protein Data Bank (RCSB PDB).

6. The protein nanoparticle according to claim 3 wherein ND1 and/or ND2 is a tetrameric coiled-coil.

7. The protein nanoparticle according to claim 6 wherein ND1 and/or ND2 is the tetrameric coiled coil from tetrabrachion (1FE6) or the tetrameric coiled coil from tetrabrachion (1FE6) which contains an amino acid modification and/or is shortened at either or both ends, wherein the tetrameric coiled coil from tetrabrachion is indicated according to the pdb entry numbering of the RCSB Protein Data Bank (RCSB PDB).

8. The protein nanoparticle according to claim 1 wherein the domains SHB1 and/or SHB2 are each independently selected from the group consisting of 4I2L, 3W19, 3VTQ, 3VU5, 3VU6, 3VTP, 3VGY, 3VH7, 3VGX, 3VIE, 3RRR, 3RRT, 3KPE, 3G7A, 3F4Y, 3F50, 1ZV8, 4NJL, 4NSM, 4JF3, 4JGS, 4JPR, 2OT5, 3CP1, 3CYO, 2IEQ, 1JPX, 1JQ0, 1K33, 1K34, 5J0J, 5J0I, 5J0H, 5IZS, 5J73, 5J2L, 5J0L, 5J0K, and 5J10, or wherein the domains SHB1 and/or SHB2 are each independently selected from the group consisting of 4I2L, 3W19, 3VTQ, 3VU5, 3VU6, 3VTP, 3VGY, 3VH7, 3VGX, 3VIE, 3RRR, 3RRT, 3KPE, 3G7A, 3F4Y, 3F50, 1ZV8, 4NJL, 4NSM, 4JF3, 4JGS, 4JPR, 20T5, 3CP1, 3CYO, 2IEQ, 1JPX, 1JQ0, 1K33, 1K34, 5J0J, 5J0I, 5J0H, 5IZS, 5J73, 5J2L, 5J0L, 5J0K, and 5J10 which contain an amino acid modification and/or is shortened at either or both ends, wherein each SHB is indicated according to the pdb entry numbering of the RCSB Protein Data Bank (RCSB PDB).

9. The protein nanoparticle according to claim 1 wherein B is selected from a protein or peptide which induces an immune response against cancer cells, a protein or peptide which induces an immune response against infectious diseases, protein or peptide which induces an immune response against allergens, protein or peptide which induces an immune response for the treatment of a human disease.

10. The protein nanoparticle according to claim 1 wherein B is selected from the group of trimeric surface glycoproteins of enveloped viruses of Class I.

11. The protein nanoparticle according to claim 1 wherein B is selected from the group consisting of trimeric surface glycoproteins of influenza virus A and B (HA), HIV (gp160), Ebola (GP), Marburg (GP), RSV (F-protein), CMV (gB protein), HSV (gB protein), SARS (S-protein) and MERS (S-protein).

12. The protein nanoparticle according to claim 1 wherein the multitude of building blocks of formula (Ia) or formula (Ib) is co-assembled with the multitude of building blocks of formula (IIa) or formula (IIb), wherein at least one of X2 and Y2 of formula (IIa) and/or formula (IIb) is a full length flagellin or a flagellin comprising only two or three domains.

13. A composition comprising a protein nanoparticle according to claim 1.

14. A monomeric building block of formula (Ia) or (Ib) 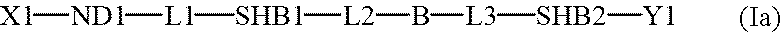 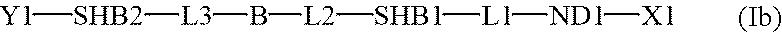
consisting of a continuous chain comprising an oligomerization domain ND1, a linker L1, a domain SHB1, a linker L2, a domain B comprising a loop region, a linker L3, a domain SHB2, and further substituents X1 and Y1, wherein
ND1 is a peptide or protein that comprises oligomers (ND1)ₘ of m subunits ND1,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X1 is absent or a peptide or protein sequence comprising 1 to 1000 amino acids that may be further substituted,
Y1 is absent or a peptide or protein sequence comprising 1 to 1000 amino acids that may be further substituted, or
a monomeric building block of formula (IIa) or (IIb) 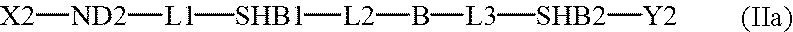 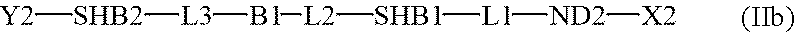
consisting of a continuous chain comprising an oligomerization domain ND2, a linker L1, a domain SHB1, a linker L2, a domain B comprising a loop region, a linker L3, a domain SHB2, and further substituents X2 and Y2, wherein
ND2 is a peptide or protein that comprises oligomers (ND2)ₘ of m subunits ND2,
SHB1 and SHB2 are independently from each other a helix of a six-helix bundle peptide or protein,
m is a figure between 2 and 10, with the proviso that m is not equal 3 and not a multiple of 3,
L1, L2 and L3 are linkers which are independently from each other a peptide bond or a peptide chain,
B is a peptide or protein comprising a loop region,
X2 is absent or a peptide or protein sequence comprising 1 to 1000 amino acids that may be further substituted,
Y2 is absent or a peptide or protein sequence comprising 1 to 1000 amino acids that may be further substituted.

15. A method of vaccinating a human or non-human animal in need thereof, comprising administering an effective amount of said protein nanoparticle of claim 1 to the human or non-human animal in need of such vaccination.


SEQUENCE LISTING (96 sequences)

SEQ ID NO: 1 - protein, 304 aa, synthetic construct
  REGION 1..304: note = HC_AD1g
MGHHHHHHKRGSWREWNAKWDEWENDWNDWREDWQAWRDDWAYWTLTWRYGELYSRLAEIETLLRGIVQQQQQLLDVVKRQQEMLRLVVWGTKNLQARVAEAWCVDQRRTLEVFKELSKINPSAILSAIYNKPIAARFMGDVLGLASCVTINQTSVKVLRDMNVKESPGRCYSRPVVIFNFARSEYVQYGQLGEDNEILLGNHRTEECQLPSLKIFIAGNSAYEYVDYLFKRMIDDGGEGPYRVCSMAQGTDLIRFERNIVCTGTDEDKQEWEHKIRFLEANISESLEQAQIQQEKNMYELQKL

SEQ ID NO: 2 - protein, 12 aa, synthetic construct
  REGION 1..12: note = His-tag
MGHHHHHHKRGS

SEQ ID NO: 3 - protein, 36 aa, synthetic construct
  REGION 1..36: note = Pentameric coiled coil ND1
WREWNAKWDEWENDWNDWREDWQAWRDDWAYWTLTW

SEQ ID NO: 4 - protein, 13 aa, synthetic construct
  REGION 1..13: note = Linker, L1
RYGELYSRLAEIE

SEQ ID NO: 5 - protein, 38 aa, synthetic construct
  REGION 1..38: note = SHB from HIV gp41
TLLRGIVQQQQQLLDVVKRQQEMLRLVVWGTKNLQARV

SEQ ID NO: 6 - protein, 164 aa, synthetic construct
  REGION 1..164: note = CMV-trimeric B-cell epitope
AEAWCVDQRRTLEVFKELSKINPSAILSAIYNKPIAARFMGDVLGLASCVTINQTSVKVLRDMNVKESPGRCYSRPVVIFNFARSEYVQYGQLGEDNEILLGNHRTEECQLPSLKIFIAGNSAYEYVDYLFKRMIDDGGEGPYRVCSMAQGTDLIRFERNIVCT

SEQ ID NO: 7 - protein, 35 aa, synthetic construct
  REGION 1..35: note = SHB from HIV gp41
QEWEHKIRFLEANISESLEQAQIQQEKNMYELQKL

SEQ ID NO: 8 - protein, 53 aa, synthetic construct
  REGION 1..53: note = tryptophan-zipper pentamerization
SSNAKWDQWSSDWQTWNAKWDQWSNDWNAWRSDWQAWKDDWARWNQRWDNWAT

SEQ ID NO: 9 - protein, 38 aa, Human immunodeficiency virus
TLFRGIVQQQQQLLDVVKRQQEMLRLTVWGTKNLQARV

SEQ ID NO: 10 - protein, 846 aa, Human immunodeficiency virus
MSGKIQLLVAFLLTSACLIYCTKYVTVFYGVPVWKNASIPLFCATKNRDTWGTIQCLPDNDDYQEIPLNVTEAFDAWDNIVTEQAVEDVWNLFETSIKPCVKLTPLCVTMNCNASTESAVATTSPSGPDMINDTDPCIQLNNCSGLREEDMVECQFNMTGLELDKKKQYSETWYSKDVVCESDNSTDRKRCYMNHCNTSVITESCDKHYWDAMRFRYCAPPGFVLLRCNDTNYSGFEPNCSKVVASTCTRMMETQPSTWLGFNGTRAENRTYIYWHGRDNRTIISLNKYYNLTILCRRPENKTVVPITLMSGRRFHSQKIINKKPRQAWCRFKGEWREAMQEVKQTLVKHPRYKGTNDTNKINFTAPEKDSDPEVAYMWTNCRGEFLYCNMTWFLNWVENKTGQQHNYVPCHIEQIINTWHKVGKNVYLPPREGELSCESTVTSIIANIDVDGDNRTNITFSAEVAELYRLELGDYKLVEVTPIGFAPTAEKRYSSAPGRHKRGVLVLGFLGFLTTAGAAMGAASLTLSAQSRTLFRGIVQQQQQLLDVVKRQQEMLRLTVWGTKNLQARVTAIEKYLADQARLNSWGCAFRQVCHTTVPWVNDTLTPEWNNMTWQEWEHKIRFLEANISESLEQAQIQQEKNMYELQKLNSWDVFGNWFDLTSWIKYIQYGVMIVVGIVALRIVIYVVQMLSRLRKGYRPVFSSPPGYIQQIHIHKDWEQPDREETEEDVGNDVGSRSWPWPIEYIHFLIRLLIRLLTRLYNSCRDLLSRLYLILQPLRDWLRLKAAYLQYGCEWIQEAFQALARVTRETLTSAGRSLWGALGRIGRGILAVPRRIRQGAEIALL

SEQ ID NO: 11 - protein, 136 aa, Human cytomegalovirus
AEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMID

SEQ ID NO: 12 - protein, 23 aa, Human cytomegalovirus
PYRVCSMAQGTDLIRFERNIVCT

SEQ ID NO: 13 - protein, 5 aa, synthetic construct
  REGION 1..5: note = Connector
DGGEG

SEQ ID NO: 14 - protein, 906 aa, synthetic construct
  REGION 1..906: note = CMV full-length gB
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEENV

SEQ ID NO: 15 - protein, 6 aa, synthetic construct
  REGION 1..6: note = Linker
GTDEDK

SEQ ID NO: 16 - protein, 688 aa, synthetic construct
  REGION 1..688: note = F34-HAPR-HIVlong
MGNNMTWQEWEHKIRFLEANISESLEQAQIQQEKNMYELQKLNSWDVFGAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKGSTLSAQVRTLLAGIVQQQQQLLDVVKRQQEMLRLVVWGVKNLQARVTAIEKYLKRLRAALQGGAIINETADDIVYRLTVIIDDRYESLKNLITLRADRLEMIINDNVSTILASIGGDEGDEGDEAREGHHHHHHHHHHGS

SEQ ID NO: 17 - protein, 47 aa, synthetic construct
  REGION 1..47: note = SHB of F34-HAPR-HIVlong
NNMTWQEWEHKIRFLEANISESLEQAQIQQEKNMYELQKLNSWDVFG

SEQ ID NO: 18 - protein, 494 aa, synthetic construct
  REGION 1..494: note = B of F34-HAPR-HIVlong
DADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNNAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESK

SEQ ID NO: 19 - protein, 52 aa, synthetic construct
  REGION 1..52: note = SHB of F34-HAPR-HIVlong
TLSAQVRTLLAGIVQQQQQLLDVVKRQQEMLRLVVWGVKNLQARVTAIEKYL

SEQ ID NO: 20 - protein, 11 aa, synthetic construct
  REGION 1..11: note = L1 of F34-HAPR-HIVlong
KRLRAALQGGA

SEQ ID NO: 21 - protein, 50 aa, synthetic construct
  REGION 1..50: note = ND1 of F34-HAPR-HIVlong
IINETADDIVYRLTVIIDDRYESLKNLITLRADRLEMIINDNVSTILASI

SEQ ID NO: 22 - protein, 26 aa, synthetic construct
  REGION 1..26: note = X1 of F34-HAPR-HIVlong
GGDEGDEGDEAREGHHHHHHHHHHGS

SEQ ID NO: 23 - protein, 636 aa, synthetic construct
  REGION 1..636: note = F3-HAPR
MGNNMTWQEWEHKIRFLEANISESLEQAQIQQEKNMYELQKLNSWDVFGAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKGSTLSAQVRTLLAGIVQQQQQLLDVVKRQQEMLRLVVWGVKNLQARVTAIEKYLKRLRAALQGGGDEGDEGDEAREGHHHHHHHHHHGS

SEQ ID NO: 24 - protein, 226 aa, synthetic construct
  REGION 1..226: note = 4TVP-1ENV
MGDKHHHHHHHHHHKDGSDKGSWEEWNARWDEWENDWNDWREDWQAWRDDWARWRATWMGGRLLSRLERLERRNVEARQLLSGIVQQQNNLLRAIEAQQHLLQLTVWVKLTPLCVTLQCTNVTNNITDDMRGELKNCSFNMTTELRDKKQKVYSLFYRLDVVQINENQGNRSNNSNKEYRLINCNTSAIMEWDREINNYTSLIHSLIEESQNQQEKNEQELLELDK

SEQ ID NO: 25 - protein, 22 aa, synthetic construct
  REGION 1..22: note = X1 of 4TVP-1ENV
MGDKHHHHHHHHHHKDGSDKGS

SEQ ID NO: 26 - protein, 36 aa, synthetic construct
  REGION 1..36: note = ND1 of 4TVP-1ENV
WEEWNARWDEWENDWNDWREDWQAWRDDWARWRATW

SEQ ID NO: 27 - protein, 17 aa, synthetic construct
  REGION 1..17: note = L1 of 4TVP-1ENV
MGGRLLSRLERLERRNV

SEQ ID NO: 28 - protein, 32 aa, synthetic construct
  REGION 1..32: note = SHB of 4TVP-1ENV
EARQLLSGIVQQQNNLLRAIEAQQHLLQLTVW

SEQ ID NO: 29 - protein, 82 aa, synthetic construct
  REGION 1..82: note = B of 4TVP-1ENV
VKLTPLCVTLQCTNVTNNITDDMRGELKNCSFNMTTELRDKKQKVYSLFYRLDVVQINENQGNRSNNSNKEYRLINCNTSAI

SEQ ID NO: 30 - protein, 37 aa, synthetic construct
  REGION 1..37: note = SHB of 4TVP-1ENV
MEWDREINNYTSLIHSLIEESQNQQEKNEQELLELDK

SEQ ID NO: 31 - protein, 67 aa, synthetic construct
  REGION 1..67: note = Combination of ACZ06517.1, ABW95233.1 and AFU33883.1
VKLTPLCVTLICKDTTNSTGTMKNCSFSVTTELRDKKQKVYALFYKLDIVPIETGEYRLINCNTSVI

SEQ ID NO: 32 - protein, 32 aa, synthetic construct
  REGION 1..32: note = SHB of 4TVP
QARNLLSGIVQQQSNLLRAPEAQQHLLKLTVW

SEQ ID NO: 33 - protein, 36 aa, synthetic construct
  REGION 1..36: note = SHB of 4TVP
LQWDKEISNYTQIIYGLLEESQNQQEKNEQDLLALD

SEQ ID NO: 34 - protein, 32 aa, synthetic construct
  REGION 1..32: note = SHB of T865/T651 pair
QARQLLSGIVQQQNNLLRAIEAQQHLLQLTVW

SEQ ID NO: 35 - protein, 37 aa, synthetic construct; identical to SEQ ID NO: 30
  REGION 1..37: note = SHB of T865/T651 pair

SEQ ID NO: 36 - protein, 10 aa, synthetic construct
  REGION 1..10: note = Coiled-coil sequence
ELYSRLAEIE

SEQ ID NO: 37 - protein, 277 aa, synthetic construct
  REGION 1..277: note = Flagellin D0-D1
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 38 - protein, 279 aa, synthetic construct
  REGION 1..279: note = Flagellin D0-D1
MAQVINTNSLSLLTQNNLNRSQSALGTAIERLSSGLRINSARDDAAGQAIANRFTANIRGLTQASRNANDGISIAQTTEGALNEINNNLQRVRELAVQSANSTNSQSDLDSIQAEITQRLNEIDRVSGQTQFNGVRVLAQDNTLTIQVGANDGETIDIDLRQINSQTLGLDQLNVQQKYKDGDKGDDKTENPLQRIDAALAQVDALRSDLGAVQNRFNSAITNLGNTVNNLSEARSRIEDSDYATEVSNMSRAQILQQAGTSVLAQANQVPQNVLSLLR

SEQ ID NO: 39 - protein, 23 aa, synthetic construct
  REGION 1..23: note = Flagellin linker
QLNVQQKYKDGDKGDDKTENPLQ

SEQ ID NO: 40 - protein, 31 aa, synthetic construct
  REGION 1..31: note = 4PN8
  SITE 1: note = misc_feature - Xaa can be any naturally occurring amino acid
XGKIEQILQKIEKILQKIEWILQKIEQILQG

SEQ ID NO: 41 - protein, 31 aa, synthetic construct
  REGION 1..31: note = 4PND
  SITE 1: note = misc_feature - Xaa can be any naturally occurring amino acid
XGNILQKIENILKKIENILWKIENILQKIEG

SEQ ID NO: 42 - protein, 52 aa, Simian rotavirus
IEKQMDRVVKEMRRQLEMIDKLTTRAIEAVELLKRIYDKLTVQTTGEIDMTK

SEQ ID NO: 43 - protein, 45 aa, Mus musculus
MDLAPQMLRELQETNAALQDVRELLRQQVKEITFLKNTVMECDAC

SEQ ID NO: 44 - protein, 45 aa, Mus musculus; identical to SEQ ID NO: 43

SEQ ID NO: 45 - protein, 45 aa, Mus musculus; identical to SEQ ID NO: 43

SEQ ID NO: 46 - protein, 45 aa, Mus musculus; identical to SEQ ID NO: 43

SEQ ID NO: 47 - protein, 330 aa, Thermotoga maritima
GSHMDFEIEVMNYSIEEFREFKTTDVESVLPFRDSSTPTWINITGIHRTDVVQRVGEFFGIHPLVLEDILNVHQRPKVEFFENYVFIVLKMFTYDKNLHELESEQVSLILTKNCVLMFQEKIGDVFDPVRERIRYNRGIIRKKRADYLLYSLIDALVDDYFVLLEKIDDEIDVLEEEVLERPEKETVQRTHQLKRNLVELAATIWPLREVLSSLYRDVPPLIEKETVPYFRDVYDHTIQIADTVETFRDIVSGLLDVYLSSVSNKTNEVMKVLTIIATIFMPLTFIAGIYGMNFEYMPELRWKWGYPVVLAVMGVIAVIMVVYFKKKKWL

SEQ ID NO: 48 - protein, 330 aa, Thermotoga maritima; identical to SEQ ID NO: 47

SEQ ID NO: 49 - protein, 53 aa, Rotavirus subgroup 1
MIEQQMDRIVKEMRRQLEMIDKLTTREIEQIELLKRIHDNLITRPVNVIDMSM

SEQ ID NO: 50 - protein, 45 aa, Mus musculus; identical to SEQ ID NO: 43

SEQ ID NO: 51 - protein, 46 aa, Rattus norvegicus
MDLAPQMLRELQETNAALQDVRELLRQQVKEITFLKNTVMECDACG

SEQ ID NO: 52 - protein, 46 aa, Rattus norvegicus; identical to SEQ ID NO: 51

SEQ ID NO: 53 - protein, 56 aa, Escherichia coli
SSNAKFDQFSSDFQTFNAKFDQFSNDFNAFRSDFQAFKDDFARFNQRFDNFATKYR

SEQ ID NO: 54 - protein, 52 aa, Homo sapiens
MEKVQYLTRSAIRRASTIEMPQQARQKLQNLFINFCLILICLLLICIIVMLL

SEQ ID NO: 55 - protein, 52 aa, Homo sapiens; identical to SEQ ID NO: 54

SEQ ID NO: 56 - protein, 53 aa, Escherichia coli; identical to SEQ ID NO: 8

SEQ ID NO: 57 - protein, 52 aa, Staphylothermus marinus
GSIINETADDIVYRLTVIIDDRYESLKNLITLRADRLEMIINDNVSTILASG

SEQ ID NO: 58 - protein, 80 aa, Human immunodeficiency virus type 1
  REGION 42..43: note = misc_feature - Xaa can be any naturally occurring amino acid
QQQNNLLRAIEAQQHLLQLTVWGIKQLQARILAVERYLKDQXXMTWETWEREIENYTKQIYKILEESQEQQDRNEKDLLE

SEQ ID NO: 59 - protein, 69 aa, Human immunodeficiency virus type 1
  SITE 1: note = misc_feature - Xaa can be any naturally occurring amino acid
  SITE 40: note = misc_feature - Xaa can be any naturally occurring amino acid
XNNLLRAIEAQQHLLQLTVWGIKQLQARILAVERYLKDQXWNEMTWMEWEREIENYTKLIYKILEESQE

SEQ ID NO: 60 - protein, 77 aa, Human immunodeficiency virus type 1
  SITE 1: note = misc_feature - Xaa can be any naturally occurring amino acid
  REGION 38..39: note = misc_feature - Xaa can be any naturally occurring amino acid
  SITE 77: note = misc_feature - Xaa can be any naturally occurring amino acid
XSGIVQQQNNLLRAIEAQQHLLQLTVWGIKQLQARILXXMTWETWEREIENYTKQIYKILEESQEQQDRNEKDLLEX

SEQ ID NO: 61 - protein, 62 aa, Human immunodeficiency virus type 1
  REGION 39..40: note = misc_feature - Xaa can be any naturally occurring amino acid
NNLLRAIEAQQHLLQLTVWGIKQLQARILAVERYLKDQXXWEEWDKKIEEYTKKIEELIKKS

SEQ ID NO: 62 - protein, 64 aa, Human immunodeficiency virus type 1
  REGION 39..40: note = misc_feature - Xaa can be any naturally occurring amino acid
NNLLRAIEAQQHLLQLTVWGIKQLQARILAVERYLKDQXXMTWEEWDKKIEEYTKKIEELIKKS

SEQ ID NO: 63 - protein, 81 aa, Human immunodeficiency virus type 1
  SITE 1: note = misc_feature - Xaa can be any naturally occurring amino acid
  REGION 43..44: note = misc_feature - Xaa can be any naturally occurring amino acid
  SITE 81: note = misc_feature - Xaa can be any naturally occurring amino acid
XQQQNNLLRAIEAQQHLLQLTVWGIKQLQARILAVERYLKDQXXMTWMEWDREINNYTSLIHSLIEESQNQQEKNEQELLX

SEQ ID NO: 64 - protein, 92 aa, Human immunodeficiency virus type 1
GSAMADIGSEFSGIVQQQNNLLRAIEAQQHLLQLTVWGIKQLQARILAVERYLKSGGRGGVEWNEMTWMEWEREIENYTKLIYKILEESQEQ

SEQ ID NO: 65 - protein, 92 aa, Human immunodeficiency virus type 1; identical to SEQ ID NO: 64

SEQ ID NO: 66 - protein, 72 aa, Human immunodeficiency virus type 1
  SITE 1: note = misc_feature - Xaa can be any naturally occurring amino acid
  SITE 40: note = misc_feature - Xaa can be any naturally occurring amino acid
XNNLLRAIEAQQHLLQLTVWGIKQLQARILAVERYLKDQXQIWNNMTWMEWDREINNYTSLIHSLIEESQNQ

SEQ ID NO: 67 - protein, 74 aa, Human immunodeficiency virus type 1
  SITE 1: note = misc_feature - Xaa can be any naturally occurring amino acid
  SITE 38: note = misc_feature - Xaa can be any naturally occurring amino acid
XSGIVQQQNNLLRAIEAQQHLLQLTVWGIKQLQARILXSWETWEREIENYTRQIYRILEESQEQQDRNERDLLE

SEQ ID NO: 68 - protein, 458 aa, Human respiratory syncytial virus
QNITEEFYQSTCSAVSKGYLSALRTGWYTSVITIELSNIKENKCNGTDAKVKLIKQELDKYKNAVTELQLLMQSTPATNNRARRAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGLEVLFQ

SEQ ID NO: 69 - protein, 458 aa, Human respiratory syncytial virus; identical to SEQ ID NO: 68

SEQ ID NO: 70 - protein, 90 aa, Human respiratory syncytial virus
HLEGEVNKIKSALLSTNKAVVSLSNGVSVLTSKVLDLKNYIDKQLLPIVNKVFPSDEFDASISQVNEKINQSLAFIRKSDELLHNVNAGK

SEQ ID NO: 71 - protein, 74 aa, Human immunodeficiency virus type 1
  SITE 51: note = misc_feature - Xaa can be any naturally occurring amino acid
  SITE 55: note = misc_feature - Xaa can be any naturally occurring amino acid
  SITE 58: note = misc_feature - Xaa can be any naturally occurring amino acid
  SITE 69: note = misc_feature - Xaa can be any naturally occurring amino acid
  SITE 72: note = misc_feature - Xaa can be any naturally occurring amino acid
SGIVQQQNNLLRAIEAQQHLLQLTVWGIKQLQARILTTWEAWDRAIAEYAXRIEXLIXAAQEQQEKNEXALXEL

SEQ ID NO: 72 - protein, 78 aa, Human immunodeficiency virus type 1
  SITE 1: note = misc_feature - Xaa can be any naturally occurring amino acid
  REGION 38..39: note = misc_feature - Xaa can be any naturally occurring amino acid
  SITE 78: note = misc_feature - Xaa can be any naturally occurring amino acid
XSGIVQQQNNLLRAIEAQQHLLQLTVWGIKQLQARILXXTTWEAWDRAIAEYAARIEALIRAAQEQQEKNEAALRELX

SEQ ID NO: 73 - protein, 78 aa, Human immunodeficiency virus type 1
  SITE 1: note = misc_feature - Xaa can be any naturally occurring amino acid
  REGION 38..40: note = misc_feature - Xaa can be any naturally occurring amino acid
  SITE 44: note = misc_feature - Xaa can be any naturally occurring amino acid
  SITE 47: note = misc_feature - Xaa can be any naturally occurring amino acid
  SITE 54: note = misc_feature - Xaa can be any naturally occurring amino acid
  SITE 58: note = misc_feature - Xaa can be any naturally occurring amino acid
  SITE 61: note = misc_feature - Xaa can be any naturally occurring amino acid
  SITE 72: note = misc_feature - Xaa can be any naturally occurring amino acid
  SITE 75: note = misc_feature - Xaa can be any naturally occurring amino acid
  SITE 78: note = misc_feature - Xaa can be any naturally occurring amino acid
XSGIVQQQNNLLRAIEAQQHLLQLTVWGIKQLQARILXXXTWEXWDXAIAEYAXRIEXLIXAAQEQQEKNEXALXELX

SEQ ID NO: 74 - protein, 86 aa, Human coronavirus
NQKQIANQFNKAISQIQESLTTTSTALGKLQDVVNQNAQALNTLVKQLSSDISGINASVVNIQKEIDRLNEVAKNLNESLIDLQEL

SEQ ID NO: 75 - protein, 131 aa, Human coronavirus
SGITQQVLSENQKLIANKFNQALGAMQTGFTTTNEAFQKVQDAVNNNAQALSKLASELSNTFGAISASIGDIIQRLDVLEQSGGRGGSIPNFGSLTQINTTLLDLTYEMLSLQQVVKALNESYIDLKELGN

SEQ ID NO: 76 - protein, 87 aa, Streptococcus pyogenes
DGEDAQKRAQIQKREELLSALIDGTSRLENKQFPYPGSTGLDDTYMNSLIQYLQERKQIEDKWRASLLKGIQDHVLDSAWSHPQFEK

SEQ ID NO: 77 - protein, 104 aa, Mason-Pfizer monkey virus
GSSTGAAGLGVSITQYTKLSHQLISDVQAISSTIQDLQDQVDSLAEVVLQNRRGLDLLTAEQGGICLALQEKCSFYANKSGIVRDKIKNLQDDLERRRRQLIDN

SEQ ID NO: 78 - protein, 105 aa, Xenotropic murine leukemia virus
SSGLVGSGTALVATKQFEQLQAAIHTDLGALEKSVSALEKSLTSLSEVVLQNRRGLDLLFLKEGGLCAALKEECSFYADHTGVVRDSMAKLRERLNQRQRLFESG

SEQ ID NO: 79 - protein, 95 aa, Avian leukosis virus
MAHHHHHHVDDDDKMSENLYFQGTANLTTSLLGDLLDDVTSIRHAVLQNRAAIDFLLLAHGHGCEDVAGMCSFNLSDQSESIQKKFQLMKEHVNK

SEQ ID NO: 80 - protein, 68 aa, Human immunodeficiency virus type 1
SGIVQQQNDLLRAIEAQQHLLQLTVWGIKQLQARSGGRGGWMEWDREINNYTSLIHSLIEESQNQQEK

SEQ ID NO: 81 - protein, 86 aa, Human immunodeficiency virus type 1
TLTVQARQLLSGIVQQQNDLLRAIEAQQHLLQLTVWGIKQLQARSGGRGGWMEWDREINNYTSLIHSLIEESQNQQEKNEQELLEL

SEQ ID NO: 82 - protein, 86 aa, Human immunodeficiency virus type 1
TLTVQARQLLSGIVQQQNDLLRAIEAQQHLLQLTVWGIKQLQARSGGRGGWMEWDREINNYTSLIHSLIEKSQNQQEKNEQELLEL

SEQ ID NO: 83 - protein, 109 aa, Human coronavirus
KAINNIVASFSSVNDAITQTAEAIHTVTIALNKIQDVVNQQGSALNHLTSQLRHNFQSGGRGSGRGGNLTYLNLSSELKQLEAKTASLFQTTVELQGLIDQINSTYVDL

SEQ ID NO: 84 - protein, 84 aa, Simian immunodeficiency virus
RTLLAGIVQQQQQLLDVVKRQQELLRLTVWGTKNLQTRVTSGGRGGWQEWERKVDFLEENITALLEEAQIQQEKNMYELQKLNS

SEQ ID NO: 85 - protein, 84 aa, Simian immunodeficiency virus
RTSLAGIVQQQQQLLDLVTRQQELLRLTVWGIKNLQTRVTSGGRGGWQEWKRKVDFLEENITALLEEAQIQQEKNMYELQKLNS

SEQ ID NO: 86 - protein, 68 aa, Human immunodeficiency virus type 1
SGIVQQQNNLLRAIEAQQHLLQLTVWGIKQLQARSGGRGGWMEWDREANNYTSLIHSLIEESQNQQEK

SEQ ID NO: 87 - protein, 68 aa, Human immunodeficiency virus type 1
SGIVQQQNNLLRAIEAQQHLLQLTVWGIKQLQARSGGRGGWMEWDREINNYTSLAHSLIEESQNQQEK

SEQ ID NO: 88 - protein, 79 aa, synthetic construct
  REGION 1..79: note = 5J0J
GSHMGTKYKIKETLKRLEDSLRELRRILEELKEMLERLEKNPDKDVIVEVLKVIVKAIEASVENQRISAENQKALAESD

SEQ ID NO: 89 - protein, 79 aa, synthetic construct
  REGION 1..79: note = 5J0I
GSHMGTKYELRRALEELEKALQELREMLRKLKESLEELKKNPSEDALVRNNELIVEVLRVIVEVLSIIARVLEINARSD

SEQ ID NO: 90 - protein, 79 aa, synthetic construct
  REGION 1..79: note = 5J0H
GSHMGTKYELRRALEELEKALRELKKSLDELERSLEELEKNPSEDALVENNRLNVENNKIIVEVLRIIAEVLKINAKSD

SEQ ID NO: 91 - protein, 95 aa, synthetic construct
  REGION 1..95: note = 5IZS
SEELRAVADLQRLNIELARKLLEAVARLQELNIDLVRKTSELTDEKTIREEIRKVKEESKRIVEEAEEEIRRAKEESRYIADESRGSLEHHHHHH

SEQ ID NO: 92 - protein, 81 aa, synthetic construct
  REGION 1..81: note = 5J73
GSHMGTSDYIIEQIQRDQEEARKKVEEAEERLERVKEASKRGVSSDQLLDLIRELAEIIEELIRIIRRSNEAIKELIKNQS

SEQ ID NO: 93 - protein, 81 aa, synthetic construct
  REGION 1..81: note = 5J2L
GSHMGSEDYKLREAQRELDKQRKDTEEIRKRLKEIQRLTDERTSTADELIKELREIIRRLQEQSEKLREIIEELEKIIRKR

SEQ ID NO: 94 - protein, 141 aa, synthetic construct
  REGION 1..141: note = 5JOL
SLMEKAARAAKELSRESARAAKELADSNAKAAEDLMREIARSSSSERLLELMAEAIRELQKQAAESIADSQRLVVEAIIRLAEAVKQGASEKEIDEIVEEAKKRLEELAERSRQENKKIIDRAKYEMDEESGSLEHHHHHH

SEQ ID NO: 95 - protein, 80 aa, synthetic construct
  REGION 1..80: note = 5J0K
GSHMGTRTEIIRELERSLREQEELAKRLKELLRELERLQREGSSDEDVRELLREIKELVEEIEKLAREQKYLVEELKRQD

SEQ ID NO: 96 - protein, 81 aa, synthetic construct
  REGION 1..81: note = 5J10
GSHMGTDTDELLRLAKEQAELLKEIKKLVEEIARLVKEIQEDPSDELLKTLAELVRKLKELVEDMERSMKEQLYIIKKQKS